(19) United States
(12) Patent Application Publication
(10) Pub. No.: US 2021/0128596 A1
(43) Pub. Date: May 6, 2021

(54) COMPOSITIONS AND METHODS FOR TREATING SEPTIC CARDIOMYOPATHY

(71) Applicant: UNIVERSITY OF PENNSYLVANIA, Philadelphia, PA (US)

(72) Inventor: Melpo CHRISTOFIDOU-SOLOMIDOU, Eagleville, PA (US)

(21) Appl. No.: 16/651,243

(22) PCT Filed: Sep. 27, 2018
(86) PCT No.: PCT/US2018/053199

§ 371 Date: Mar. 26, 2020

Related U.S. Application Data
(60) Provisional application No. 62/564,173, filed on Sep. 27, 2017.

(51) Int. Cl. A61K 31/7034; A61K 9/00; A61P 9/00

(52) U.S. Cl. CPC A61K 31/7034; A61P 9/00; A61K 9/0053

(57) ABSTRACT

Methods and compositions are provided for treating sepsis-associated cardiac dysfunction, specifically sepsis-induced cardiomyopathy, and for protecting the heart from sepsis-associated dysfunction and improving cardiac function in subjects having sepsis. These methods include administering compositions comprising secoisolariciresinol diglucoside (SDG) or related compounds, obtained from natural sources, such as flaxseed, or generated synthetically.

### CROSS-REFERENCE TO RELATED APPLICATIONS

This application is a U.S. national stage application under 37 U.S.C. 371 of PCT International Application PCT/US2018/053199, filed Sep. 27, 2018, which claims benefit of U.S. Provisional Patent Application Ser. No. 62/564,173, filed Sep. 27, 2017, the priority date of which is hereby claimed, the contents of each of which is hereby incorporated by reference in its entirety.

### FIELD OF THE INVENTION

The invention relates to the use of secoisolariciresinol diglucoside (SDG), obtained from natural sources, such as flaxseed, or generated synthetically (synthetic SDG is also referred to herein as LGM2605), other active components in flaxseed, secoisolariciresinol (SECO), enterodiol (ED), and enterolactone (EL), as well as stereoisomers of the foregoing, metabolites of the foregoing, degradants of the foregoing, and analogs of the foregoing, for treating sepsis-associated cardiac dysfunction, such as sepsis-induced cardiomyopathy, and for protecting the heart from sepsis-associated dysfunction and improving cardiac function in subjects having sepsis.

### BACKGROUND OF THE INVENTION

Sepsis is the manifestation of the immune and inflammatory response to infection that may ultimately result in multi-organ failure. 20 to 30 million people become septic each year and over 8 million die. A patient with sepsis is five times more likely to die than a patient who suffered a heart attack or stroke. Sepsis is also the most common cause of death in intensive care units worldwide. Sepsis affects all ages from neonatal through to the elderly and critically ill; it is often diagnosed too late for treatment to be effective. The basic pathophysiologic defect in sepsis, causing functional abnormalities in many organ systems, remains elusive. Myocardial dysfunction is a well-described complication of severe sepsis, also referred to as septic cardiomyopathy or sepsis-induced cardiomyopathy. In sepsis-induced cardiomyopathy both right and left ventricles can dilate, contractile function may decrease, and ventricular compliance is reduced (Kumar et al., (2000) Crit Care Clin. 16:251-287). In addition, severe depression of ejection fraction has been demonstrated in some patients with sepsis despite normal or elevated cardiac index (Parker et al., (1984) Ann Int Med 100:483-490).

Although myocardial dysfunction in sepsis has been the focus of many investigations, its etiology remains unclear. Possible underlying causes of sepsis-induced cardiomyopathy include, inter alia, increased inflammation, oxidative stress, impaired ATP production within cardiomyocytes, and, possibly, impaired adrenergic signaling in the heart. Given the significant link between sepsis and mortality, a need clearly exists for improved methodologies for the treatment and resolution of sepsis.

### SUMMARY OF THE INVENTION

In one aspect, provided herein are methods for treating or preventing sepsis-induced cardiomyopathy in a subject in need thereof, comprising: administering to the subject an effective amount of secoisolariciresinol diglucoside (SDG), an analog thereof, a stereoisomer thereof, or a combination thereof, thereby treating sepsis-induced cardiomyopathy in the subject.

In another aspect, provided herein are methods for maintaining cardiac function in a subject having sepsis, comprising: administering to the subject an effective amount of secoisolariciresinol diglucoside (SDG), an analog thereof, a stereoisomer thereof, or a combination thereof, thereby preserving cardiac function in the subject.

In a further aspect, provided herein are methods for improving cardiac contractility and/or cardiomyocyte mitochondrial function in a subject having sepsis, comprising: administering to the subject an effective amount of secoisolariciresinol diglucoside (SDG), an analog thereof, a stereoisomer thereof, or a combination thereof, thereby improving cardiac contractility in the subject.

In an additional aspect, provided herein are methods for reducing oxidative stress in cardiomyocytes of a subject having sepsis, comprising: administering to the subject an effective amount of secoisolariciresinol diglucoside (SDG), an analog thereof, a stereoisomer thereof, or a combination thereof, thereby reducing oxidative stress in the subject.

In a further aspect, provided herein are methods for treating septic cardiomyopathy in a subject in need thereof, the method comprising: administering to said subject an effective amount of secoisolariciresinol diglucoside (SDG), thereby treating said septic cardiomyopathy in said subject.

In a further aspect, provided herein are methods for treating sepsis-associated cardiac dysfunction in a subject in need thereof, the method comprising: administering to said subject an effective amount of secoisolariciresinol diglucoside (SDG), thereby treating said sepsis-associated cardiac dysfunction in said subject.

In a further aspect, provided herein are methods for improving mitochondrial function in cardiac myocytes of a subject in need thereof, the method comprising: administering to said subject an effective amount of secoisolariciresinol diglucoside (SDG), thereby improving said mitochondrial function in cardiac myocytes of said subject.

### BRIEF DESCRIPTION OF THE DRAWINGS

The following drawings form part of this specification and are included to further demonstrate certain aspects of this disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1. Shows a schematic summarizing possible mechanisms underlying sepsis-induced cardiac dysfunction.

FIG. 2. SDG prevents septic cardiac dysfunction. (A) An M-mode echocardiogram of mice treated with SDG either 2 hours prior to cecal ligation puncture (CLP) or 6 hours after CLP or a sham procedure. (B) A graph showing percent ejection fraction (EF, left) and fractional shortening (FS, right) for mice receiving different treatments.

FIG. 3. SDG increases adenylyl cyclase (AC) expression in vivo. (A) A western blot showing AC V/VI expression in mice after undergoing CLP or a sham procedure with and without SDG treatment. (B) A graph showing densitometric analysis of the western blot in (A), expressed as AC/GAPDH ratio.

FIG. 4. SDG increases cAMP activity in AC16 cells at 12 hours in non-stimulated conditions. The graph shows cAMP levels in AC16 cells that were treated with SDG, Liposaccharide (LPS) or both either without stimulation, or in the presence of forskolin or isoprotenerol.

FIG. 5. SDG augments isoproterenol-stimulated protein kinase A (PKA) activation in AC16 cells at 12 hours but not in the disease state. (A) A graph showing PKA activity levels in AC16 cells that were treated with SDG, Liposaccharide (LPS) or both either without stimulation, or in the presence of isoprotenerol. (B) A graph showing pairwise comparison of PKA activity levels in AC16 cells in the absence or presence of isoprotenerol under various conditions.

FIG. 6. SDG suppresses the LPS-mediated increase in mitochondrial superoxide generation in AC16 cells. (A) Fluorescent micrographs showing mitosox red staining of AC16 cells that were either left untreated (control), or 12 hours after LPS treatment alone or with SDG. (B) A graphical representation of the data shown in (A).

FIG. 7. SDG prevents LPS-mediated decrease in mitochondrial number in AC16 cells. (A) Fluorescent micrographs showing staining of AC16 cells that were either left untreated (control), or 12 hours after LPS treatment alone or with SDG. (B) A graphical representation of the data shown in (A).

FIG. 8. SDG treatment restores the sepsis-induced changes in mRNA levels of fusion and fission markers. (A) Expression levels fold change of fusion and fission markers in untreated mice (control) and in mice treated with LPS with and without SDG treatment. (B) Expression levels fold change of fusion and fission markers in untreated mice (control) and in mice that underwent CLP with and without SDG treatment administered 6 hours after CLP treatment.

FIG. 9. SDG treatment of LPS-stimulated AC16 cells tends to increase the expression of MCU and MICU1. (A) Expression levels of Mitochondrial Calcium Uniporter (MCU) and Mitochondrial Calcium Uptake 1 (MICU1) in untreated mice (control) and in mice treated with LPS with and without SDG treatment. (B) A western blot showing MCU expression in untreated mice and in mice treated with LPS with and without SDG treatment. (C) A graph showing densitometric analysis of the western blot in (B), expressed as fold change of MCU expression. (D) A western blot showing MICU1 expression in untreated mice and in mice treated with LPS with and without SDG treatment. (E) A graph showing densitometric analysis of the western blot in (D), expressed as fold change of MICU1 expression.

FIG. 10. SDG treatment of septic mice increases the protein levels of MCU in the heart tissue. (A) A western blot showing MCU expression in untreated mice (control) and in mice that underwent CLP, with and without SDG treatment. (B) A graph showing densitometric analysis of the western blot in (A), expressed as fold change of MCU expression. (C) A western blot showing MICU1 expression in untreated mice and in mice that underwent CLP, with and without SDG treatment. (D) A graph showing densitometric analysis of the western blot in (C), expressed as fold change of MICU1 expression.

FIG. 11. SDG increases the oxygen consumption rate of cardiomyocytes in septic mice. (A) The seahorse analysis plot of untreated mice (control) and mice that underwent CLP, with and without SDG treatment. (B) graphs presenting comparison of various mitochondrial respiration parameters under different conditions.

FIG. 12: Establishment of septic cardiac function using the cecal ligation and puncture model (CLP) (A) Representative M-mode echocardiograms after CLP surgery. (B) Graph of ejection fraction (EF) and fractional shortening (FS), n=5 mice, One-way ANOVA analysis *p:<0.05 vs baseline. **p:<0.01 vs baseline. (C) Body temperature of mice 12 hours after sham or CLP surgery. n=3-4 mice. (D) Graph of DP/dt maximum of mice 12 hours post-sham and CLP surgery. n=3 mice. (E) Inflammatory cytokine gene expression in ventricular tissue of mice 12 hours post-sham and CLP surgery, n=4-5 mice per group. *p<0.05, **p<0.01, ***p<0.001 vs Sham by t-test.

FIG. 13. LGM2605 prevents septic cardiac dysfunction in C57BL/6 mice following CLP surgery without reducing inflammatory cytokines. (A-B) Representative M-mode echocardiograms (A), ejection fraction (EF) and fractional shortening (FS) of C57BL/6 mice treated with LGM2605 6 hrs post-CLP and monitored for 12 hrs after the surgery. Sham: n=9, Sham+6 hrs SDG: n=4, CLP: n=12, CLP+6 hrs SDG: n=12, **P<0.01 vs Sham, ###P<0.001 vs CLP, $P<0.05 vs Sham+LGM2605 by ANOVA with Bonferroni post-test (C) Immunoblotting and densitometric analysis of phosphorylated and total IkBα from ventricular tissue of mice 12 hours post-surgery. (D) Cardiac mRNA expression and (E) plasma levels of cytokines 12-hours post-surgery, n=4-5 mice per group. *P<0.05, **P<0.01, ***P<0.001 by ANOVA with Bonferroni post-test.

FIG. 14: LGM2605-mediated improvement in cardiac function is not associated with altered β-AR signaling. (A-B) LVdP/dtmax as an index of cardiac contractility and LVdP/dtmin as an index of myocardial relaxation to increasing doses of isoproterenol in mice that underwent sham surgery, CLP and combined CLP and LGM2605 treatment (6 h post-CLP), at 12 hrs timepoint. n=3 mice per group, **P<0.01, ***P<0.001 versus sham at corresponding timepoints, #P<0.05 versus baseline, ##P<0.01 versus baseline, ###P<0.001 versus baseline, +P<0.05 and ++P<0.01 versus 0.1 ng isoproterenol, @ P<0.05 versus 0.5 ng isoproterenol, by ANOVA with Bonferroni post-test. (C) Density of β adrenergic receptors using radio ligand binding assay, n=4-5 mice per group. ***P<0.001 by ANOVA with Bonferroni post-test.

FIG. 15: LGM2605 alleviates mitochondrial oxidative stress without altering fatty acid and glucose metabolism-related gene expression program. (A) Expression of glucose metabolism-related genes in ventricular tissue 12 hours after surgery, n=4 mice per group. (B) Plasma glucose levels 12 hours after surgery, n=4 mice per group. (C) Expression of lipid metabolism-related genes in ventricular tissue 12 hours after surgery, n=4-5 mice per group. (D) Plasma triglyceride content 12 hours after surgery, n=4 mice per group. (E) Representative fluorescence microscopy images and quantification of Mitosox Red staining in AC16 cells stimulated with LPS and LPS+LGM2605 for 12 hours, n=250. (F) Representative confocal microscopy images and quantification of DHE staining intensity in ventricular tissue of mice 12 hours after surgery, n=3 mice per group. (G) Antioxidant-related gene expression in ventricular tissue 12 hours after surgery, n=4-5 mice per group. *P<0.05, ** P<0.01, ***P<0.001 vs Control/Sham ^{###}p<0.001 vs LPS/CLP by ANOVA with Bonferroni post-test.

FIG. 16: LGM2605 increases mitochondrial abundance (A) Representative fluorescent images and (B) quantification of mitotracker signal in AC16 cells treated with LPS, LPS+LGM2605 or Vehicle for 12 hours, n=250 cells per group. (C) Representative fluorescent images and (D) quantification of mitochondria detection by Mitotracker Red from adult cardiomyocytes isolated from mice 12 hrs after surgery, n=150 cells. (E) Graph of mitochondrial biogenesis-related gene expression from ventricular tissue of mice 12 hrs after surgery, n=4-5 mice per group. (F) Fusion and fission gene expression-related markers in ventricular tissue of mice 12 hrs after surgery, n=4 mice per group. (G-H) Representative LC3B and densitometric quantification of LC3BII/LC3BI ratio, n=8-9 mice per group. (I) rtPCR analysis of autophagy related genes. *p<0.05, **p<0.01, ***p<0.001 vs Sham/Saline, #p<0.05, ###p<0.001 vs LPS/CLP by ANOVA+Bonferroni post-test.

FIG. 17: LGM2605 increases mitochondrial calcium uptake in isolated primary cardiomyocytes from septic mice. (A-C) Mitochondrial calcium uptake after a single bolus of calcium in permeabilized adult cardiomyocytes isolated from mice at 12 hrs after surgery, n=3 mice per group. #P<0.05 vs CLP by ANOVA with Bonferroni post-HOC test. (D) MCU, MICU1 gene expression from ventricular tissue of mice 12 hrs after sham surgery, CLP and CLP surgery followed by treatment with LGM2605 at 6 hrs post-CLP, n=4-5 mice. (E) Immunoblots and densitometry analysis of MCU, MICU1 western blots, in relative units, from ventricular tissue 12 hrs after surgery, n=8-9 mice.

FIG. 18: LGM2605 increases oxygen consumption in cardiomyocytes isolated from septic mice. (A-G) Oxygen consumption rate in isolated adult cardiomyocytes 12 hrs after sham surgery, CLP surgery and CLP followed by treatment with LGM2605 at 6 hrs post-CLP measured using Seahorse XF Mito Stress kit. Graphs of basal respiration (B), respiration for ATP production (C), maximal respiration (D), and spare capacity (E). **P<0.01, ***P<0.001 vs Sham, ##P<0.01 vs CLP by ANOVA with Bonferroni post-test. Mice used in the seahorse experiment and wells analyzed: 3 sham mice (total 34 wells), 4 CLP mice (total 30 wells), 4 CLP+SDG mice (total 38 wells).

FIG. 19: LGM2605 preserves mitochondrial membrane potential in LPS stimulated AC16 cardiomyocytes. (A) Representative images and (B) fluorescence intensity quantification from AC16 cells treated for 12 hours with LPS and LGM2605 with and without uncoupling agent 2,4-DNP (50 μM). *p<0.05, ***P<0.001 vs Vehicle; ##P<0.01 vs LPS, $$$P<0.001 vs LGM2605 by ANOVA+Bonferroni post-hoc analysis. (C) Graphical model of the proposed mechanism by which LGM2605 alleviates oxidative stress, increases mitochondrial respiration, and restores cardiac systolic function. Figure was produced using Servier Medical Art.

FIG. 20: (A-B) Representative M-mode echocardiograms, ejection fraction (EF) and fractional shortening (FS) of C57BL/6 mice treated with LGM2605 2 hrs prior to CLP and monitored for 12 hrs after the surgery. Sham: n=9, CLP: n=12, CLP+2 hrs prior SDG: n=4. ***p<0.001 vs Sham, ##p<0.01 vs CLP, by ANOVA with Bonferroni post-test (C) Echocardiography analysis for female C57BL/6 mice 12 hours after CLP surgery.

FIG. 21: (A) Cardiac IL-1β, IL-6, and TNFα mRNA levels of mice 6 hours post-sham surgery, CLP or CLP+LGM2605 dosed 2 hours before surgery, n=3-4 mice per group. *P<0.05, **P<0.01 vs Sham 6h by ANOVA with Bonferroni post-test. (B) Plasma glucose levels from mice at the 6 hour timepoint, n=4 mice per group. ***P<0.001 vs sham by ANOVA with Bonferroni post-test.

FIG. 22: (A) TMRM Staining of AC16 stimulated with increasing concentration of uncoupling agent 2,4-DNP. (B) Fluorescence intensity quantification of LGM2605 and DNP 2,4-DNP stimulated AC16 cells. ***p<0.001 vs 0 μM DNP, ###p<0.001 vs 10 μM DNP, $$P<0.001 VS 50 by ANOVA with Bonferroni post-test.

FIGS. 23A, 23B, and 23C show that reduced ROS generation via NOX2 inhibition improves cardiac function in sepsis.

FIG. 24 shows that LGM2605 is chemically synthesized antioxidant Secoisolariciresinol Diglucoside (SDG).

FIG. 25 illustrates a working model.

### DETAILED DESCRIPTION OF THE INVENTION

The invention relates to the use of secoisolariciresinol diglucoside (SDG), obtained from natural sources, such as flaxseed, or generated synthetically (synthetic SDG is also referred to herein as LGM2605), other active components in flaxseed, and related compounds for treating and preventing sepsis-associated cardiac dysfunction or sepsis-induced cardiomyopathy. Surprisingly and unexpectedly, the inventors have found that SDG can be used to restore and maintain cardiac function in a subject having sepsis.

Accordingly, in one embodiment, provided herein are methods for treating sepsis-induced cardiomyopathy in a subject in need thereof, comprising: administering to said subject an effective amount of secoisolariciresinol diglucoside (SDG), an analog thereof, a stereoisomer thereof, or a combination thereof. In another embodiment, provided herein are methods for preventing sepsis-induced cardiomyopathy in a subject in need thereof, comprising: administering to said subject an effective amount of secoisolariciresinol diglucoside (SDG), an analog thereof, a stereoisomer thereof, or a combination thereof.

In a further embodiment, provided herein are methods for maintaining and/or restoring cardiac function in a subject having sepsis, comprising: administering to said subject an effective amount of secoisolariciresinol diglucoside (SDG), an analog thereof, a stereoisomer thereof, or a combination thereof.

As used herein, “sepsis” refers to the systemic inflammatory response associated with infection. The “systemic inflammatory response” is the body's overwhelming response to a noxious stimulus. The characteristics of this response may include one or more of the following non-specific changes in the adult human body:
(a) fast heart rate (tachycardia, heart rate >90 beats per minute);
(b) low blood pressure (systolic <90 mmHg or MAP <65 mmHg);
(c) low or high body temperature (<36 or >38° C.);
(d) high respiratory rate (>20 breaths per minute); and
(e) low or high white blood cell count (<4 or >12 billion cells/liter).

The causes and clinical manifestations of sepsis are well known in the art and, for example, are described in detail in the US2016/0166598, which is incorporated by reference herein in its entirety.

Severe sepsis is associated with profound cardiovascular dysfunction that may include hypotension, decreased systemic resistance, altered vascular reactivity to contractile agents and/or decreased myocardial contractility. Systemic infection depresses heart function and the severity of this myocardial depression correlates with a poor prognosis. Echocardiographic studies suggest that 40% to 50% of patients with prolonged septic shock develop myocardial depression, as determined by a reduced ejection fraction.

Accordingly, in one embodiment provided herein are methods for improving cardiac contractility in a subject having sepsis, comprising: administering to said subject an effective amount of secoisolariciresinol diglucoside (SDG), an analog thereof, a stereoisomer thereof. In another embodiment, provided herein are methods for maintaining cardiac contractility in a subject having sepsis, comprising: administering to said subject an effective amount of secoisolariciresinol diglucoside (SDG), an analog thereof, a stereoisomer thereof. In another embodiment, provided herein are methods for restoring cardiac contractility in a subject having sepsis, comprising: administering to said subject an effective amount of secoisolariciresinol diglucoside (SDG), an analog thereof, a stereoisomer thereof.

In another embodiment, provided herein are methods for treating myocardial depression in a subject having sepsis comprising: administering to said subject an effective amount of secoisolariciresinol diglucoside (SDG), an analog thereof, a stereoisomer thereof. In another embodiment, provided herein are methods for preventing myocardial depression in a subject having sepsis, comprising: administering to said subject an effective amount of secoisolariciresinol diglucoside (SDG), an analog thereof, a stereoisomer thereof.

The functional symptoms of sepsis-related impairment of cardiac contractility include, without limitation, reduced ejection fraction (EF %) of both ventricles, reduced fractional shortening (FS %), increased end-diastolic volume (EDV) and end-systolic volume (ESV), and low stroke volume (SV).

Thus, in one embodiment, the compositions described herein increase ejection fraction (EF %) in a subject having sepsis. In another embodiment, the compositions described herein increase fractional shortening (FS %) in a subject having sepsis. In another embodiment, the compositions described herein reduce end-diastolic volume (EDV) in a subject having sepsis. In another embodiment, the compositions described herein reduce end-systolic volume (ESV) in a subject having sepsis. In another embodiment, the compositions described herein increase stroke volume (SV) in a subject having sepsis. In another embodiment, the compositions described herein reverse a functional symptom of sepsis-related impairment of cardiac contractility known in the art.

Without wishing to be bound by any particular theory, sepsis-related impairment of cardiac contractility is caused by sepsis-induced structural changes in the heart. The structural changes include, without limitation, myocardial edema, myocardial infiltration by immune cells (especially macrophages and neutrophils), subendocardial hemorrhage, interstitial and intracellular edema, endothelial cell edema, microcirculatory fibrin deposition, intracytoplasmic lipid accumulation in cardiomyocytes, as well as focal myofibrillar dissolution, and interstitial fibrosis. These structural changes lead to defects in heart function, specifically, profound myocardial depression, global left ventricular (LV) hypokinesia, reduced LV dilatation, abnormal ventricular relaxation, and septoapical hypokinesia.

Myocardial depression during sepsis involves a complex mix of systemic (hemodynamic) factors and genetic, molecular, metabolic, and structural alterations. Manifestations of myocardial depression include, inter alia, reduced left ventricular ejection fraction (LVEF), reduced left ventricular ejection fraction (LVEF), and cardiomyocyte necrosis and apoptosis

Accordingly, the sepsis-induced structural change in the heart treated by the compositions described herein is, in one embodiment, myocardial edema. In another embodiment, the sepsis-induced structural change in the heart treated by the compositions described herein is myocardial infiltration by immune cells. In another embodiment, the sepsis-induced structural change in the heart treated by the compositions described herein is subendocardial hemorrhage. In another embodiment, the sepsis-induced structural change in the heart treated by the compositions described herein is interstitial and intracellular edema. In another embodiment, the sepsis-induced structural change in the heart treated by the compositions described herein is endothelial cell edema. In another embodiment, the sepsis-induced structural change in the heart treated by the compositions described herein is microcirculatory fibrin deposition. In another embodiment, the sepsis-induced structural change in the heart treated by the compositions described herein is intracytoplasmic lipid accumulation in cardiomyocytes. In another embodiment, the sepsis-induced structural change in the heart treated by the compositions described herein is focal myofibrillar dissolution. In another embodiment, the sepsis-induced structural change in the heart treated by the compositions described herein is cardiomyocyte necrosis. In another embodiment, the sepsis-induced structural change in the heart treated by the compositions described herein is myofibrillar interstitial fibrosis.

In one embodiment, the sepsis-induced cardiac function defect treated by the compositions described herein is myocardial depression. In another embodiment, the sepsis-induced cardiac function defect treated by the compositions described herein is global left ventricular (LV) hypokinesia. In another embodiment, the sepsis-induced cardiac function defect treated by the compositions described herein is reduced LV dilatation. In another embodiment, the sepsis-induced cardiac function defect treated by the compositions described herein is abnormal ventricular relaxation. In another embodiment, the sepsis-induced cardiac function defect treated by the compositions described herein is septoapical hypokinesia. In another embodiment, the sepsis-induced cardiac function defect treated by the compositions described herein is reduced left ventricular ejection fraction (LVEF).

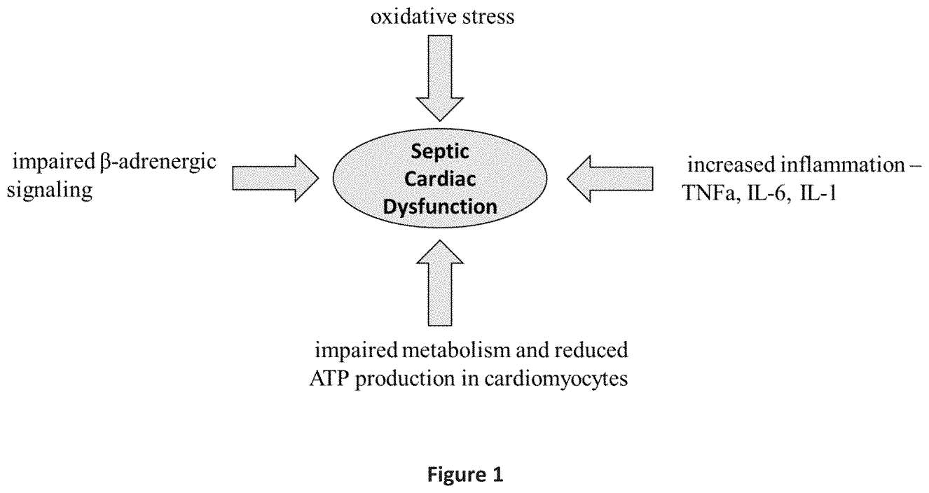

Without wishing to be bound by theory, the molecular mechanisms underlying induced cardiac dysfunction include oxidative stress, increased inflammation (mediated by TNF-α, IL-1β, and IL-6), as well as impaired metabolism and reduced ATP production in cardiomyocytes (FIG. 1). In particular, oxidative stress is induced by reactive oxygen species (ROS), which are mainly generated via mitochondrial respiration. Moreover, mitochondria themselves are thought to be the primary target of oxidative damage, specifically, increased mitochondrial permeability resulting in further release of mitochondrial ROS, such as superoxide, leading to further oxidative damage.

Accordingly, in one embodiment, provided herein are methods for reducing oxidative stress in cardiomyocytes of a subject having sepsis, comprising: administering to said subject an effective amount of secoisolariciresinol diglucoside (SDG), an analog thereof, a stereoisomer thereof.

In another embodiment, provided herein are methods for suppressing mitochondrial reactive oxygen species generation in cardiomyocytes of a subject having sepsis, comprising: administering to said subject an effective amount of secoisolariciresinol diglucoside (SDG), an analog thereof, a stereoisomer thereof. In another embodiment, provided herein are methods for suppressing mitochondrial superoxide generation in cardiomyocytes of a subject having sepsis, comprising: administering to said subject an effective amount of secoisolariciresinol diglucoside (SDG), an analog thereof, a stereoisomer thereof. In another embodiment, provided herein are methods for suppressing mitochondrial hydrogen peroxide generation in cardiomyocytes of a subject having sepsis, comprising: administering to said subject an effective amount of secoisolariciresinol diglucoside (SDG), an analog thereof, a stereoisomer thereof.

Without wishing to be bound by any particular theory, sepsis causes a defect at the mitochondrial level such that cells cannot use oxygen for energy production. Cardiomyocytes may become “functionally hypoxic” during sepsis, because even though there is adequate oxygen available, the cell cannot use the oxygen for aerobic oxidative phosphorylation. This inability to use molecular oxygen for energy production during sepsis has been termed “cytopathic hypoxia.” Cytopathic hypoxia is associated with a decrease in myocardial ATP. Given that mitochondria comprise about 30% of myocardial volume, maintaining normal mitochondrial function may be important for reducing sepsis-related oxidative stress in cardiomyocytes.

Moreover, normal mitochondrial function depends not only on proper functioning of oxidative phosphorylation, but also on preservation of mitochondrial biogenesis, which includes maintaining or increasing cellular mitochondrial mass and copy number, as well as effective removal of damaged mitochondria and unwanted mitochondrial molecules. This is accomplished through the process of mitochondrial fission and fusion, through which, the mitochondrial network is constantly remodeled. Fission and fusion increase in stress conditions, playing critical roles in removing damaged mitochondria and augmenting repair processes. Sepsis causes disruption of mitochondrial fission/fusion balance, ultimately resulting in overall decrease of mitochondrial mass and number in cardiomyocytes, ultimately resulting in cytopathic hypoxia. Thus, under septic conditions the expression of numerous fusion and fission markers is suppressed. Therefore, restoring proper expression of mitochondrial fusion and fission regulating factors will likely restore mitochondrial biogenesis, including maintaining or restoring cardiomyocyte mitochondrial mass and number and efficient removal of damaged mitochondria.

Accordingly, in one embodiment, provided herein are methods for stimulating mitochondrial biogenesis in a subject having sepsis, comprising: administering to said subject an effective amount of secoisolariciresinol diglucoside (SDG), an analog thereof, a stereoisomer thereof, or a combination thereof. In another embodiment, provided herein are methods for maintaining mitochondrial biogenesis in a subject having sepsis, comprising: administering to said subject an effective amount of secoisolariciresinol diglucoside (SDG), an analog thereof, a stereoisomer thereof, or a combination thereof. In another embodiment, provided herein are methods for restoring mitochondrial biogenesis in a subject having sepsis, comprising: administering to said subject an effective amount of secoisolariciresinol diglucoside (SDG), an analog thereof, a stereoisomer thereof, or a combination thereof.

In another embodiment, provided herein are methods for increasing the number of cardiomyocyte mitochondria in a subject having sepsis, comprising: administering to said subject an effective amount of secoisolariciresinol diglucoside (SDG), an analog thereof, a stereoisomer thereof, or a combination thereof. In another embodiment, provided herein are methods for maintaining the number of cardiomyocyte mitochondria in a subject having sepsis, comprising: administering to said subject an effective amount of secoisolariciresinol diglucoside (SDG), an analog thereof, a stereoisomer thereof, or a combination thereof. In another embodiment, provided herein are methods for preventing the decrease in the number of cardiomyocyte mitochondria in a subject having sepsis comprising: administering to said subject an effective amount of secoisolariciresinol diglucoside (SDG), an analog thereof, a stereoisomer thereof, or a combination thereof. In another embodiment, provided herein are methods for reversing of sepsis-mediated decrease in the number of cardiomyocyte mitochondria in a subject having sepsis, comprising: administering to said subject an effective amount of secoisolariciresinol diglucoside (SDG), an analog thereof, a stereoisomer thereof, or a combination thereof.

In another embodiment, provided herein are methods for stimulating fusion and fission of cardiomyocyte mitochondria in a subject having sepsis, comprising: administering to said subject an effective amount of secoisolariciresinol diglucoside (SDG), an analog thereof, a stereoisomer thereof, or a combination thereof. In another embodiment, provided herein are methods for maintaining fusion and fission of cardiomyocyte mitochondria in a subject having sepsis, comprising: administering to said subject an effective amount of secoisolariciresinol diglucoside (SDG), an analog thereof, a stereoisomer thereof, or a combination thereof. In another embodiment, provided herein are methods for reversing of sepsis-mediated decrease in fusion and fission of cardiomyocyte mitochondria in a subject having sepsis, comprising: administering to said subject an effective amount of secoisolariciresinol diglucoside (SDG), an analog thereof, a stereoisomer thereof, or a combination thereof.

In another embodiment, provided herein are methods for maintaining mitochondrial fusion/fission balance in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for restoring mitochondrial fusion/fission balance in cardiomyocytes of a subject having sepsis, comprising: administering to said subject an effective amount of secoisolariciresinol diglucoside (SDG), an analog thereof, a stereoisomer thereof, or a combination thereof.

In another embodiment, provided herein are methods for stimulating expression of mitochondrial fusion and fission-associated genes in a subject having sepsis, comprising: administering to said subject an effective amount of secoisolariciresinol diglucoside (SDG), an analog thereof, a stereoisomer thereof, or a combination thereof. In another embodiment, provided herein are methods for maintaining expression of mitochondrial fusion and fission-associated genes in a subject having sepsis, comprising: administering to said subject an effective amount of secoisolariciresinol diglucoside (SDG), an analog thereof, a stereoisomer thereof, or a combination thereof. In another embodiment, provided herein are methods for reversing of sepsis-mediated decrease of expression of mitochondrial fusion and fission-associated genes in a subject having sepsis, comprising: administering to said subject an effective amount of secoisolariciresinol diglucoside (SDG), an analog thereof, a stereoisomer thereof, or a combination thereof.

As used herein, the term “mitochondrial fusion associated gene” refers to a gene involved in regulation of mitochondrial fusion and includes, without limitation, Optic Atrophy 1 (OPA1), Mitofusin 1 (MFN1), Mitofusin 2 (MFN2), or a combination thereof.

As used herein, the term “mitochondrial fission associated gene” refers to a gene involved in regulation of mitochondrial fission and includes, without limitation, Dynamin related protein 1 (Drp1), Mitochondrial Fission 1 (FIS1), Mitochondrial Fission Factor (MFF), or a combination thereof.

Without wishing to be bound by theory, mitochondria play a key role in regulation of cell's homeostasis of calcium through specifically uptaking and transiently storing calcium (Ca²⁺). In addition, Ca²⁺ is an important factor that regulates mitochondrial effectors. Calcium enters the mitochondria primarily through the mitochondria calcium uniporter (MCU) complex in a tightly regulated process. Under septic conditions the expression of the primary MCU subunits, Mitochondrial Calcium Uniporter (MCU) and Mitochondrial Calcium Uptake 1 (MICU1) is decreased, resulting in dysregulation of mitochondrial Ca²⁺ uptake. This leads to disruption of myocardial Ca²⁺ homeostasis, which is one of the underlying causes of sepsis-induced cardiomyopathy. Thus, restoring proper expression of the MCU subunits will likely restore mitochondrial Ca²⁺ uptake and Ca²⁺ homeostasis in cardiomyocytes.

Accordingly, in one embodiment, provided herein are methods for maintaining Ca²⁺ homeostasis in cardiomyocytes in a subject having sepsis, comprising: administering to said subject an effective amount of secoisolariciresinol diglucoside (SDG), an analog thereof, a stereoisomer thereof, or a combination thereof. In another embodiment, provided herein are methods for restoring Ca²⁺ homeostasis in cardiomyocytes in a subject having sepsis, comprising: administering to said subject an effective amount of secoisolariciresinol diglucoside (SDG), an analog thereof, a stereoisomer thereof, or a combination thereof. In another embodiment, provided herein are methods for reversing sepsis-associated disruption of Ca²⁺ homeostasis in cardiomyocytes of a subject having sepsis, comprising: administering to said subject an effective amount of secoisolariciresinol diglucoside (SDG), an analog thereof, a stereoisomer thereof, or a combination thereof.

In another embodiment, provided herein are methods for stimulating mitochondrial Ca²⁺ uptake in a subject having sepsis, comprising: administering to said subject an effective amount of secoisolariciresinol diglucoside (SDG), an analog thereof, a stereoisomer thereof, or a combination thereof. In another embodiment, provided herein are methods for maintaining mitochondrial Ca²⁺ uptake in a subject having sepsis, comprising: administering to said subject an effective amount of secoisolariciresinol diglucoside (SDG), an analog thereof, a stereoisomer thereof, or a combination thereof. In another embodiment, provided herein are methods for restoring mitochondrial Ca²⁺ uptake in a subject having sepsis, comprising: administering to said subject an effective amount of secoisolariciresinol diglucoside (SDG), an analog thereof, a stereoisomer thereof, or a combination thereof. In another embodiment, provided herein are methods for reversing sepsis-associated dysregulation of mitochondrial Ca²⁺ uptake in cardiomyocytes of a subject having sepsis, comprising: administering to said subject an effective amount of secoisolariciresinol diglucoside (SDG), an analog thereof, a stereoisomer thereof, or a combination thereof.

In another embodiment, provided herein are methods for maintaining expression of mitochondrial calcium uniporter (MCU) subunits in a subject having sepsis, comprising: administering to said subject an effective amount of secoisolariciresinol diglucoside (SDG), an analog thereof, a stereoisomer thereof, or a combination thereof. In another embodiment, provided herein are methods for restoring expression of mitochondrial calcium uniporter (MCU) subunits in a subject having sepsis, comprising: administering to said subject an effective amount of secoisolariciresinol diglucoside (SDG), an analog thereof, a stereoisomer thereof, or a combination thereof. In another embodiment, provided herein are methods for stimulating expression of mitochondrial calcium uniporter (MCU) subunits in a subject having sepsis, comprising: administering to said subject an effective amount of secoisolariciresinol diglucoside (SDG), an analog thereof, a stereoisomer thereof, or a combination thereof. In another embodiment, provided herein are methods for reversing of sepsis-mediated decrease of expression of mitochondrial calcium uniporter (MCU) subunits in a subject having sepsis, comprising: administering to said subject an effective amount of secoisolariciresinol diglucoside (SDG), an analog thereof, a stereoisomer thereof, or a combination thereof.

As used herein, “mitochondrial calcium uniporter (MCU) subunits” refers to any of the genes encoding MCU complex protein components and to genes encoding protein factors that regulate MCU activity, as well as to the proteins encoded by these genes. These genes include, without limitation, Mitochondrial Calcium Uniporter, Mitochondrial Calcium Uptake 1 (MICU1), Mitochondrial Calcium Uptake 2 (MICU2), or any other functional or regulatory MCU complex component.

In one embodiment, provided herein are methods for stimulating cardiomyocyte mitochondrial function in a subject having sepsis, comprising: administering to said subject an effective amount of secoisolariciresinol diglucoside (SDG), an analog thereof, a stereoisomer thereof, or a combination thereof. Furthermore, in another embodiment, provided herein are methods for improving cardiomyocyte mitochondrial function in a subject having sepsis, comprising: administering to said subject an effective amount of secoisolariciresinol diglucoside (SDG), an analog thereof, a stereoisomer thereof, or a combination thereof. In another embodiment, provided herein are methods for maintaining cardiomyocyte mitochondrial function in a subject having sepsis, comprising: administering to said subject an effective amount of secoisolariciresinol diglucoside (SDG), an analog thereof, a stereoisomer thereof, or a combination thereof. In another embodiment, provided herein are methods for restoring cardiomyocyte mitochondrial function in a subject having sepsis, comprising: administering to said subject an effective amount of secoisolariciresinol diglucoside (SDG), an analog thereof, a stereoisomer thereof, or a combination thereof. In another embodiment, provided herein are methods for reversing of sepsis-mediated decrease of cardiomyocyte mitochondrial function in a subject having sepsis, comprising: administering to said subject an effective amount of secoisolariciresinol diglucoside (SDG), an analog thereof, a stereoisomer thereof, or a combination thereof.

Additionally, in one embodiment, provided herein are methods for stimulating cardiomyocytes oxygen consumption in a subject having sepsis, comprising: administering to said subject an effective amount of secoisolariciresinol diglucoside (SDG), an analog thereof, a stereoisomer thereof, or a combination thereof. In another embodiment, provided herein are methods for increasing cardiomyocytes oxygen consumption rate in a subject having sepsis, comprising: administering to said subject an effective amount of secoisolariciresinol diglucoside (SDG), an analog thereof, a stereoisomer thereof, or a combination thereof. In another embodiment, provided herein are methods for maintaining cardiomyocytes oxygen consumption rate in a subject having sepsis, comprising: administering to said subject an effective amount of secoisolariciresinol diglucoside (SDG), an analog thereof, a stereoisomer thereof, or a combination thereof. In another embodiment, provided herein are methods for restoring cardiomyocytes oxygen consumption rate in a subject having sepsis, comprising: administering to said subject an effective amount of secoisolariciresinol diglucoside (SDG), an analog thereof, a stereoisomer thereof, or a combination thereof. In another embodiment, provided herein are methods for reversing of sepsis-mediated decrease of cardiomyocytes oxygen consumption rate in a subject having sepsis, comprising: administering to said subject an effective amount of secoisolariciresinol diglucoside (SDG), an analog thereof, a stereoisomer thereof, or a combination thereof.

In one embodiment, provided herein are methods for increasing basal mitochondrial respiration in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for maintaining basal mitochondrial respiration in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for restoring basal mitochondrial respiration in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for reversing of sepsis-mediated decrease of basal mitochondrial respiration in cardiomyocytes of a subject having sepsis, comprising: administering to said subject an effective amount of secoisolariciresinol diglucoside (SDG), an analog thereof, a stereoisomer thereof, or a combination thereof.

Furthermore, in one embodiment, provided herein are methods for increasing maximal mitochondrial respiration in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for maintaining maximal mitochondrial respiration in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for restoring maximal mitochondrial respiration in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for reversing of sepsis-mediated decrease of maximal mitochondrial respiration in cardiomyocytes of a subject having sepsis, comprising: administering to said subject an effective amount of secoisolariciresinol diglucoside (SDG), an analog thereof, a stereoisomer thereof, or a combination thereof.

In one embodiment, provided herein are methods for stimulating ATP production in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for maintaining ATP production in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for restoring ATP production in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for reversing of sepsis-mediated decrease of ATP production in cardiomyocytes of a subject having sepsis, comprising: administering to said subject an effective amount of secoisolariciresinol diglucoside (SDG), an analog thereof, a stereoisomer thereof, or a combination thereof.

Furthermore, in one embodiment, provided herein are methods for increasing non-mitochondrial oxygen consumption in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for maintaining non-mitochondrial oxygen consumption in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for restoring non-mitochondrial oxygen consumption in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for reversing of sepsis-mediated decrease of non-mitochondrial oxygen consumption in cardiomyocytes of a subject having sepsis, comprising: administering to said subject an effective amount of secoisolariciresinol diglucoside (SDG), an analog thereof, a stereoisomer thereof, or a combination thereof.

Additionally, in one embodiment, provided herein are methods for increasing spare mitochondrial respiratory capacity in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for maintaining spare mitochondrial respiratory capacity in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for restoring spare mitochondrial respiratory capacity in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for reversing of sepsis-mediated decrease of spare mitochondrial respiratory capacity in cardiomyocytes of a subject having sepsis, comprising: administering to said subject an effective amount of secoisolariciresinol diglucoside (SDG), an analog thereof, a stereoisomer thereof, or a combination thereof.

Furthermore, provided herein are methods for decreasing proton leakage in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for maintaining low proton leakage in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for restoring low proton leakage in cardiomyocytes of a subject having sepsis, comprising: administering to said subject an effective amount of secoisolariciresinol diglucoside (SDG), an analog thereof, a stereoisomer thereof, or a combination thereof. In another embodiment, provided herein are methods for reversing of sepsis-mediated increase of proton leakage in cardiomyocytes of a subject having sepsis, comprising: administering to said subject an effective amount of secoisolariciresinol diglucoside (SDG), an analog thereof, a stereoisomer thereof, or a combination thereof.

Surprisingly and unexpectedly, the inventors of this application have found that SDG can be used to increase cAMP activity and to augment isoproterenol-stimulated polynucleotide kinase A (PKA) activation in cardiomyocytes. Without wishing to be bound by any particular theory, cAMP and PKA mediate 3-adrenergic signaling cascade that controls cardiomyocyte contraction. Disruption of 3-adrenergic signaling cascade can lead to heart block, low cardiac output (hypoperfusion), congestive heart failure, and cardiogenic shock. Increase of cAMP levels or PKA activity would stimulate 3-adrenergic signaling and thus increase cardiomyocyte contractility.

Accordingly, in one embodiment, provided herein are methods for stimulating 3-adrenergic signaling in a subject in need thereof, comprising: administering to said subject an effective amount of secoisolariciresinol diglucoside (SDG), an analog thereof, a stereoisomer thereof, or a combination thereof. In another embodiment, provided herein are methods for maintaining (3-adrenergic signaling in a subject in need thereof, comprising: administering to said subject an effective amount of secoisolariciresinol diglucoside (SDG), an analog thereof, a stereoisomer thereof, or a combination thereof. In another embodiment, provided herein are methods for increasing cardiomyocytes cAMP levels in a subject in need thereof, comprising: administering to said subject an effective amount of secoisolariciresinol diglucoside (SDG), an analog thereof, a stereoisomer thereof, or a combination thereof. In another embodiment, provided herein are methods for maintaining cardiomyocyte cAMP levels in a subject in need thereof, comprising: administering to said subject an effective amount of secoisolariciresinol diglucoside (SDG), an analog thereof, a stereoisomer thereof, or a combination thereof. In another embodiment, provided herein are methods for stimulating cardiomyocyte PKA activity in a subject in need thereof, comprising: administering to said subject an effective amount of a composition comprising isoproterenol and secoisolariciresinol diglucoside (SDG), an analog thereof, a stereoisomer thereof, or a combination thereof. In another embodiment, provided herein are methods for maintaining cardiomyocyte PKA activity in a subject in need thereof, comprising: administering to said subject an effective amount of a composition comprising isoproterenol and secoisolariciresinol diglucoside (SDG), an analog thereof, a stereoisomer thereof, or a combination thereof.

In another embodiment, provided herein are methods for improving cardiomyocyte contractility in a subject in need thereof, comprising: administering to said subject an effective amount of a secoisolariciresinol diglucoside (SDG), an analog thereof, a stereoisomer thereof, or a combination thereof. In another embodiment, provided herein are methods for improving cardiomyocyte contractility in a subject in need thereof, comprising: administering to said subject an effective amount of a composition comprising isoproterenol and secoisolariciresinol diglucoside (SDG), an analog thereof, a stereoisomer thereof, or a combination thereof.

In another embodiment, provided herein are methods for treating heart block in a subject, comprising: administering to said subject an effective amount of secoisolariciresinol diglucoside (SDG), an analog thereof, a stereoisomer thereof, or a combination thereof. In another embodiment, provided herein are methods for treating heart block in a subject, comprising: administering to said subject an effective amount of a composition comprising isoproterenol and secoisolariciresinol diglucoside (SDG), an analog thereof, a stereoisomer thereof, or a combination thereof.

In another embodiment, provided herein are methods for treating hypoperfusion in a subject, comprising: administering to said subject an effective amount of secoisolariciresinol diglucoside (SDG), an analog thereof, a stereoisomer thereof, or a combination thereof. In another embodiment, provided herein are methods for treating hypoperfusion in a subject, comprising: administering to said subject an effective amount of a composition comprising isoproterenol and secoisolariciresinol diglucoside (SDG), an analog thereof, a stereoisomer thereof, or a combination thereof.

In another embodiment, provided herein are methods for treating congestive heart failure in a subject, comprising: administering to said subject an effective amount of secoisolariciresinol diglucoside (SDG), an analog thereof, a stereoisomer thereof, or a combination thereof. In another embodiment, provided herein are methods for treating congestive heart failure in a subject, comprising: administering to said subject an effective amount of a composition comprising isoproterenol and secoisolariciresinol diglucoside (SDG), an analog thereof, a stereoisomer thereof, or a combination thereof.

In another embodiment, provided herein are methods for treating cardiogenic shock in a subject, comprising: administering to said subject an effective amount of secoisolariciresinol diglucoside (SDG), an analog thereof, a stereoisomer thereof, or a combination thereof. In another embodiment, provided herein are methods for treating cardiogenic shock in a subject, comprising: administering to said subject an effective amount of a composition comprising isoproterenol and secoisolariciresinol diglucoside (SDG), an analog thereof, a stereoisomer thereof, or a combination thereof.

2,3-bis (3-methoxy-4-hydroxybenzyl) butane-1,4-diol (secoisolariciresinol or SECO) is the primary lignan found in flaxseed. In its native state it is stored in the plant as the conjugate SDG. Flaxseed, its bioactive ingredients, and its metabolites are known in the art and described in U.S. Patent Publication Nos. 2010/0239696; 2011/0300247; and 2014/0308379; and in International Patent Publication No. WO2014/200964, each of which is incorporated by reference herein in its entirety.

SDG can be isolated from natural sources or chemically synthesized. Due to complex extraction, purification and enrichment methods to isolate secoisolariciresinol diglucoside (SDG) from natural resources, in a preferred embodiment, SDG is chemically synthesized.

Techniques for synthesizing SDG, its stereoisomers and analogs are described in Mishra O P, et al., Bioorganic & Medicinal Chemistry Letters 2013, (19):5325-5328 and in International Patent Publication No. WO2014/200964, which are hereby incorporated by reference in their entireties. For example, using the natural compounds vanillin and glucose, two enantiomers (their structures are depicted below) of SDG: SDG (S,S) and SDG (R,R), were successfully synthesized (Mishra et al., Bioorganic & Medicinal Chemistry Letters 2013, (19):5325).

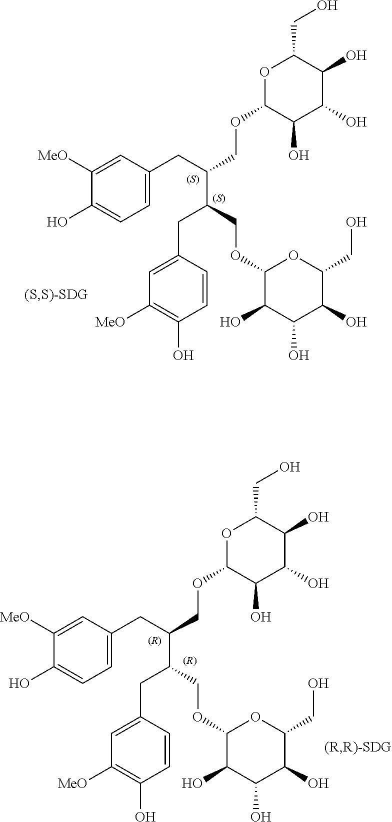

In one embodiment, the SDG administered in the methods described herein is SDG (S,S). In another embodiment, the SDG administered in the methods described herein is SDG (R,R).

SDG is metabolized in the human intestine to enterodiol (ED), and enterolactone (EL). Synthetic analogs of enterodiol and enterolactone are known (see, e.g., Eklund et al., Org. Lett. 2003, 5:491). Thus, in another aspect, other bioactive ingredients of flaxseed, their metabolites, their degradants or stereoisomers can also be used. Examples of the other bioactive ingredients of flaxseed include, for example, but not limited to, secoisolariciresinol (SECO), enterodiol (ED), enterolactone (EL), analogs thereof, isomers (including stereoisomers) thereof, or a combination thereof.

Bioactive components for use in the methods provided herein may also be chemically synthesized directly into the mammalian, readily metabolizable forms, Enterodiol (ED) or Enterolactone (EL), as is known in the art.

Thus, in one embodiment, provided herein are methods for treating sepsis-induced cardiomyopathy in a subject in need thereof, comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof.

In another embodiment, provided herein are methods for maintaining cardiac function in a subject having sepsis, comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof.

In another embodiment, provided herein are methods for improving cardiac contractility in a subject having sepsis, comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof.

In another embodiment, provided herein are methods for reducing oxidative stress in cardiomyocytes of a subject having sepsis, comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof.

In another embodiment, provided herein are methods for stimulating cardiomyocyte mitochondrial function in a subject having sepsis, comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof.

In another embodiment, provided herein are methods for stimulating β-adrenergic signaling in a subject in need thereof, comprising: administering to said subject an effective amount of at least one bioactive ingredient, wherein said bioactive ingredient comprises secoisolaricirecinol diglucoside (SDG), secoisolariciresinol (SECO), enterodiol (ED), enterolactone (EL), metabolites thereof, degradants thereof, analogs thereof, stereoisomers thereof, or a combination thereof.

In another aspect, flaxseed extract can be used. Techniques for extracting and purifying SDG are known in the art and described in U.S. Pat. No. 5,705,618, which is incorporated herein by reference in its entirety.

Thus, in one embodiment, provided herein are methods for treating sepsis-induced cardiomyopathy in a subject in need thereof, comprising: administering to said subject an effective amount of a flaxseed extract. In another embodiment, provided herein are methods for maintaining cardiac function in a subject having sepsis, comprising: administering to said subject an effective amount of a flaxseed extract. In another embodiment, provided herein are methods for improving cardiac contractility in a subject having sepsis, comprising: administering to said subject an effective amount of a flaxseed extract. In another embodiment, provided herein are methods for reducing oxidative stress in cardiomyocytes of a subject having sepsis, comprising: administering to said subject an effective amount of a flaxseed extract. In another embodiment, provided herein are methods for improving cardiomyocyte mitochondrial function in a subject having sepsis, comprising: administering to said subject an effective amount of a flaxseed extract. In another embodiment, provided herein are methods for stimulating β-adrenergic signaling in a subject in need thereof, comprising: administering to said subject an effective amount of a flaxseed extract.

A “metabolite” is a substance produced by metabolism or by a metabolic process. For example, a metabolite of SDG is EL or ED. A “degradant” is a product of the breakdown of a molecule, such as SDG, into smaller molecules. It will be appreciated by one skilled in the art that a metabolite or a degradant may be a chemically synthesized equivalent of a natural metabolite or degradant.

An “analog” is a compound whose structure is related to that of another compound. The analog may be a synthetic analog.

In another aspect, the invention relates to a pharmaceutical composition. “Pharmaceutical composition” refers to an effective amount of an active ingredient, e.g., (S,S)-SDG (R,R)-SDG, meso-SDG, SDG, SECO, EL, ED and analogs thereof, together with a pharmaceutically acceptable carrier or diluent.

The compositions described herein may include a “therapeutically effective amount.” A “therapeutically effective amount” refers to an amount effective, at dosages and for periods of time needed, to achieve the desired therapeutic result. A therapeutically effective amount may vary according to factors such as the disease state, age, sex, and weight of the individual, and the ability of the composition to elicit a desired response in the individual. A therapeutically effective amount is also one in which toxic or detrimental effects of the molecule are outweighed by the therapeutically beneficial effects.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used herein includes both one and more than one such excipient.

The pharmaceutical compositions can be administered to a subject by any suitable method known to a person skilled in the art, such as orally, parenterally, transmucosally, transdermally, intramuscularly, intravenously, intra-dermally, subcutaneously, intra-peritonealy, intra-ventricularly, intra-cranially, intra-vaginally, intra-tumorally, or bucally. Controlled release may also be used by embedding the active ingredient in an appropriate polymer which may then be inserted subcutaneously, intratumorally, bucally, as a patch on the skin, or vaginally. Coating a medical device with the active ingredient is also covered.

In some embodiments, the pharmaceutical compositions are administered orally, and are thus formulated in a form suitable for oral administration, i.e., as a solid or a liquid preparation. Suitable solid oral formulations include tablets, capsules, pills, granules, pellets and the like. Suitable liquid oral formulations include solutions, suspensions, dispersions, emulsions, oils and the like. In some embodiments, the active ingredient is formulated in a capsule. In accordance with this embodiment, the compositions described herein comprise, in addition to the active compound and the inert carrier or diluent, drying agent, in addition to other excipients, as well as a gelatin capsule.

In some embodiments, the pharmaceutical compositions are administered by intravenous, intra-arterial, or intra-muscular injection of a liquid preparation. In some embodiments, the pharmaceutical composition is a liquid preparation formulated for oral administration. In some embodiments, the pharmaceutical composition is a liquid preparation formulated for intravaginal administration. Suitable liquid formulations include solutions, suspensions, dispersions, emulsions, oils and the like. In some embodiments, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In some embodiments, the pharmaceutical compositions are administered intra-muscularly and are thus formulated in a form suitable for intra-muscular administration. In some embodiments, the pharmaceutical compositions are administered intra-bucally and are thus formulated in a form suitable for buccal administration.

In some embodiments, the pharmaceutical compositions are administered topically to body surfaces and are thus formulated in a form suitable for topical administration. Suitable topical formulations include gels, ointments, creams, lotions, drops, controlled release polymers and the like. For topical administration, the flaxseed, its bioactive ingredient, or a metabolite thereof is prepared and applied as a solution, suspension, or emulsion in a physiologically acceptable diluent with or without a pharmaceutical carrier.

In some embodiments, the pharmaceutical compositions provided herein are controlled-release compositions, i.e. compositions in which the flaxseed, its bioactive ingredient, or a metabolite thereof is released over a period of time after administration. Controlled- or sustained-release compositions include formulation in lipophilic depots (e.g. fatty acids, waxes, oils). In other embodiments, the composition is an immediate-release composition, i.e. a composition in which all the flaxseed, its bioactive ingredient, or a metabolite thereof is released immediately after administration.

In some embodiments, compositions for use in the methods provided herein are administered at a therapeutic dose once per day. In some embodiments, the compositions are administered once every two days, twice a week, once a week, or once every two weeks.

In one embodiment, (S,S)-SDG (R,R)-SDG, (S,R)-SDG (R,S)-SDG, meso-SDG, SECO, EL, ED or an analog thereof may be administered at a dose of 0.1 ng/kg to 500 mg/kg. In another embodiment, (S,S)-SDG (R,R)-SDG, (S,R)-SDG (R,S)-SDG, meso-SDG, SECO, EL, ED or an analog thereof may be administered at a concentration of about 1 nanomolar (nM) to about 1 molar (M). In another embodiment, (S,S)-SDG (R,R)-SDG, (S,R)-SDG (R,S)-SDG, meso-SDG, SECO, EL, ED or an analog thereof may be administered at a concentration from about 25 μM to about 250 μM.

The treatment regimen with (S,S)-SDG (R,R)-SDG, (S,R)-SDG (R,S)-SDG, meso-SDG, SDG, SECO, EL, ED or an analog thereof may range from a single administration to several days, months, years, or indefinitely. In one embodiment, treatment regimen with (S,S)-SDG (R,R)-SDG, (S,R)-SDG (R,S)-SDG, meso-SDG, SDG, SECO, EL, ED or an analog thereof comprises daily administration over one week. In another embodiment, treatment regimen with (S,S)-SDG (R,R)-SDG, (S,R)-SDG (R,S)-SDG, meso-SDG, SDG, SECO, EL, ED or an analog thereof comprises daily administration over two weeks. In another embodiment, treatment regimen with (S,S)-SDG (R,R)-SDG, (S,R)-SDG (R,S)-SDG, meso-SDG, SDG, SECO, EL, ED or an analog thereof comprises daily administration over three weeks. In another embodiment, treatment regimen with (S,S)-SDG (R,R)-SDG, (S,R)-SDG (R,S)-SDG, meso-SDG, SDG, SECO, EL, ED or an analog thereof comprises daily administration over one month. In another embodiment, treatment regimen with (S,S)-SDG (R,R)-SDG, (S,R)-SDG (R,S)-SDG, meso-SDG, SDG, SECO, EL, ED or an analog thereof comprises daily administration over two months. In another embodiment, treatment regimen with (S,S)-SDG (R,R)-SDG, (S,R)-SDG (R,S)-SDG, meso-SDG, SDG, SECO, EL, ED or an analog thereof comprises daily administration over three months. In another embodiment, treatment regimen with (S,S)-SDG (R,R)-SDG, (S,R)-SDG (R,S)-SDG, meso-SDG, SDG, SECO, EL, ED or an analog thereof comprises daily administration over six months.

As used herein, “treating” may refer to either therapeutic treatment or prophylactic or preventative measures, where the object is to prevent or lessen the targeted pathologic condition or disorder as described herein, or both. Therefore, compositions for use in the methods provided herein may be administered to a subject at risk of developing a pathologic condition or disorder and before said pathologic condition or disorder develops. In some cases, the compositions for use in the methods provided herein may be administered to a subject after a pathologic condition or disorder develops. Thus, treating a condition as described herein may refer to preventing, inhibiting, reversing, or suppressing the condition in a subject.

Furthermore, as used herein, the terms “treat” and “treatment” refer to therapeutic treatment, as well prophylactic or preventative measures, where the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already having a pathologic condition or disorder or those who are at risk of developing a pathologic condition or disorder.

As used herein, the term “maintaining” means to preserve or keep in a state or condition corresponding to absence of a disease or pathology and encompasses preventing a decline, lapse or cessation from that state or condition.

As used herein, the term “preventing” may refer to stopping, hindering, or suppressing a disease, disorder, or a symptom of a disease or disorder, through some action before the symptoms or consequences of the disease or disorder manifest themselves, or before a patient is exposed to conditions which may trigger the disease or disorder.

The term “subject” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In addition to humans, the subject may include dogs, cats, pigs, cows, sheep, goats, horses, buffalo, ostriches, guinea pigs, rats, mice, birds (e.g., parakeets) and other wild, domesticated or commercially useful animals (e.g., chicken, geese, turkeys, fish). The term “subject” does not exclude an individual that is normal in all respects. The term “subject” includes, but is not limited to, a human in need of therapy for, or susceptible to, a condition or its sequelae.

Thus, in one embodiment, the compositions described herein are administered prior to the subject's developing sepsis-induced cardiomyopathy or a cardiac disease or disorder associated with disruption of β-adrenergic signaling. In another embodiment, the compositions described herein are administered following the subject's developing sepsis-induced cardiomyopathy or a cardiac disease or disorder associated with disruption of β-adrenergic signaling.

By “administration prior to” is meant administration of a composition of the invention in a therapeutically effective amount before the subject may develop sepsis-induced cardiomyopathy e.g. through a medical procedure, (e.g., 4 months prior, 3 months prior, 2 months prior, 1 month prior, 4 weeks prior, 3 weeks prior, 2 weeks prior, 1 week prior, 6 days prior, 5 days prior, 4 days prior, 3 days prior, 2 days prior, 1 day prior, less than 24 hours prior (e.g., less than 23, 20, 19, 18, 17, 16, 15, 10, 9, 8, 7, 6, 5, 4, 3, 2 hours, or 1 hour).

Examples of subjects who may develop sepsis, and sepsis-induced cardiomyopathy include, but are not limited to, patients who display systemic inflammatory response syndrome (SIRS), multiple organ dysfunction syndrome (MODS), patients who are exposed to infection, patients having tumors or degenerative disease, patients who have suffered a trauma or an injury, or patients who underwent a medical procedure resulting in a controlled injury.

As used herein, the term “infection” refers to any infection, whether local or systemic, including, but not limited to, viral, bacterial, fungal and parasitic infections, that affects animals.

As used herein, the term “trauma” refers to any injury (wound or burn) to a cell, organ, tissue or whole body. The external agent causing the injury may be physical or mechanical force, such as that caused by rapid acceleration or deceleration, blast, waves, crush, in impact of penetration by a projectile. The external agent causing the injury may be also a chemical agent such as smoke, chemical irritants, or chemical or biological toxins.

As used herein, the term “controlled injury” refers to damage to tissues and organs incurred in the course of medical procedures, such as that caused by invasive or non-invasive surgery, needle placement, wound management, or intubation.

Embodiments of the methods described herein also encompass co-administration of at least one other agent that improves cardiac dysfunction (e.g., ascorbic acid or isoproterenol) with secoisolariciresinol diglucoside (SDG) for the treatment of sepsis.

Any patent, patent application publication, or scientific publication, cited herein, is incorporated by reference herein in its entirety.

In the following examples below, numerous specific details are set forth in order to provide a thorough understanding of the invention. However, it will be understood by those skilled in the art that this invention may be practiced without these specific details. In other instances, well-known methods, procedures, and components have not been described in detail so as not to obscure this invention. Thus these examples should in no way be construed, as limiting the broad scope of the invention.

### EXAMPLES

### Example 1

### SDG Prevents Septic Cardiac Dysfunction

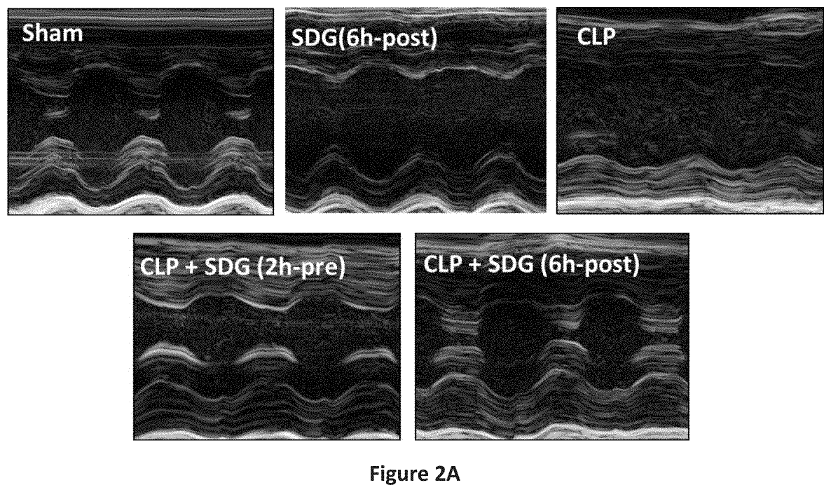

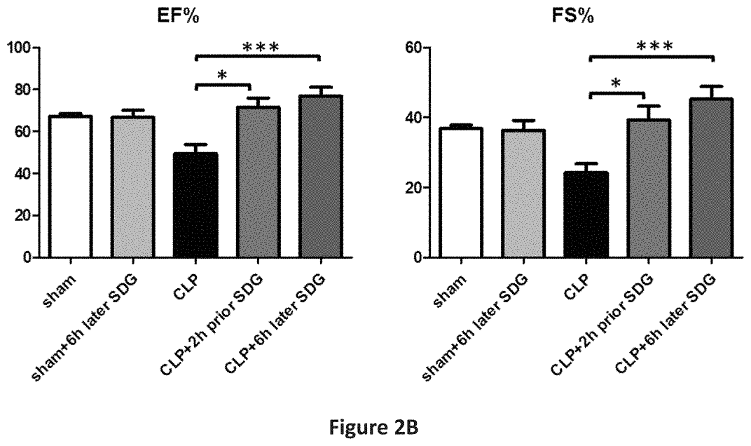

Mice were either sham treated or subjected to cecal ligation puncture (CLP). SDG was administered either 2 hours prior to CLP or 6 hours following CLP. Echocardiograms from each group of mice was collected (FIG. 2A) and ejection fraction (EF, left) and fractional shortening (FS, right) was determined. As shown in FIG. 2B, SDG treatment either before or after CLP restores EF and to pre-CLP levels.

### Example 2

### SDG Increases Adenylyl Cyclase (AC) Expression In Vivo

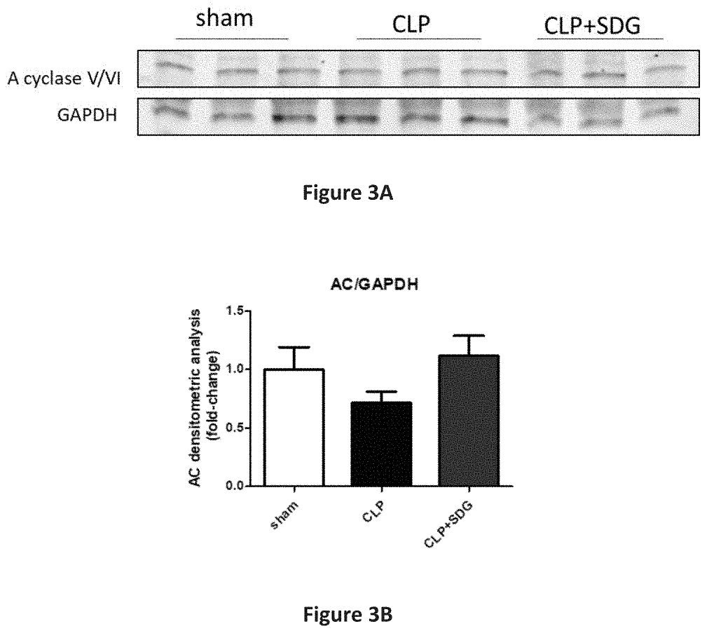

Mice were either sham treated (n=3) or subjected to cecal ligation puncture (CLP, n=6). SDG was administered to three mice following CLP. FIG. 3A shows a western blot of adenylyl cyclase V/VI expression in each mouse. The graphical representation of the data averaged across each group (FIG. 3B) shows that SDG treatment restores cyclase V/VI expression pre-CLP levels.

### Example 3

### SDG Increases cAMP Activity in AC16 Cells in Non-Stimulated Conditions

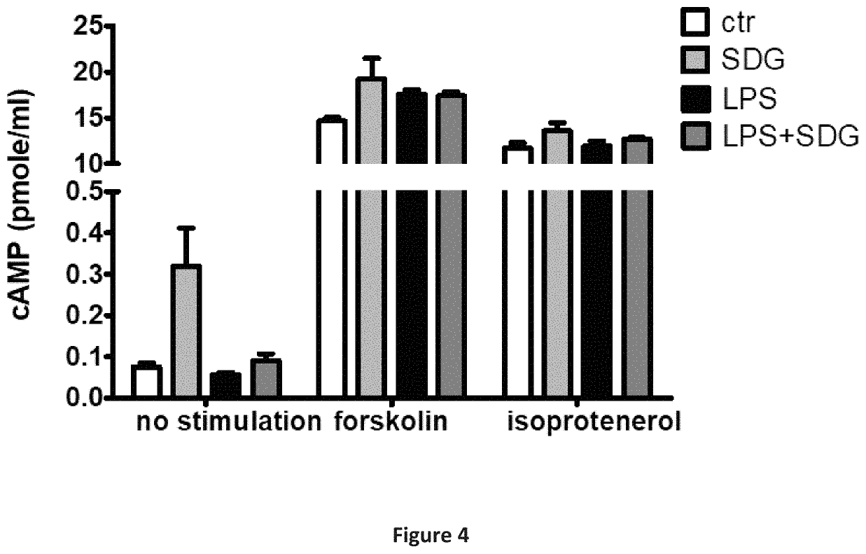

AC16 cells were either left unstimulated or stimulated with either forskolin or isoproterenol for 12 hours and then treated with SDG, Liposaccharide (LPS) or both. While SDG treatment increased cAMP levels, this increase was abolished in the presence of LPS (FIG. 4). In addition, the SDG-dependent increase of cAMP levels appears to be insignificant in the presence of forskolin or isoproterenol-dependent cAMP levels increase.

### Example 4

### SDG Augments Isoproterenol-Stimulated Protein Kinase a (PKA) Activation in AC16 Cells but not in the Disease State

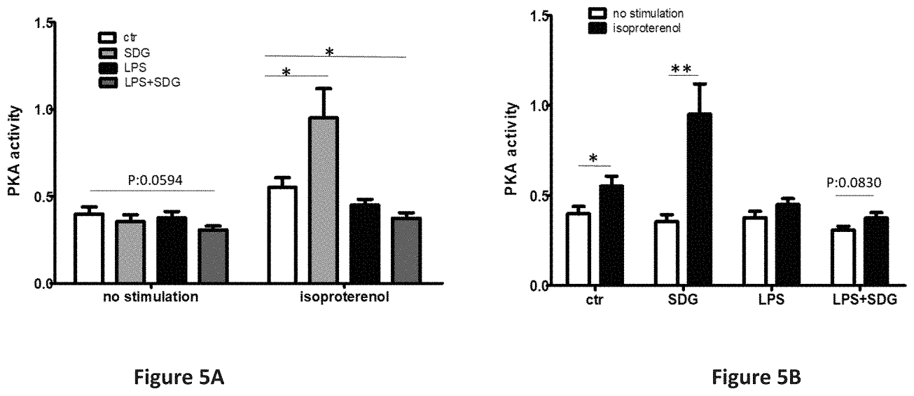

AC16 cells were either left untreated or treated with SDG, Liposaccharide (LPS), or both. While SDG treatment increased protein kinase A (PKA) activity, and the increase was amplified by isoproterenol, this stimulation was abolished in the presence of LPS (FIG. 5).

### Example 5

### SDG Suppresses the LPS-Mediated Increase in Mitochondrial Superoxide Generation in AC16 Cells

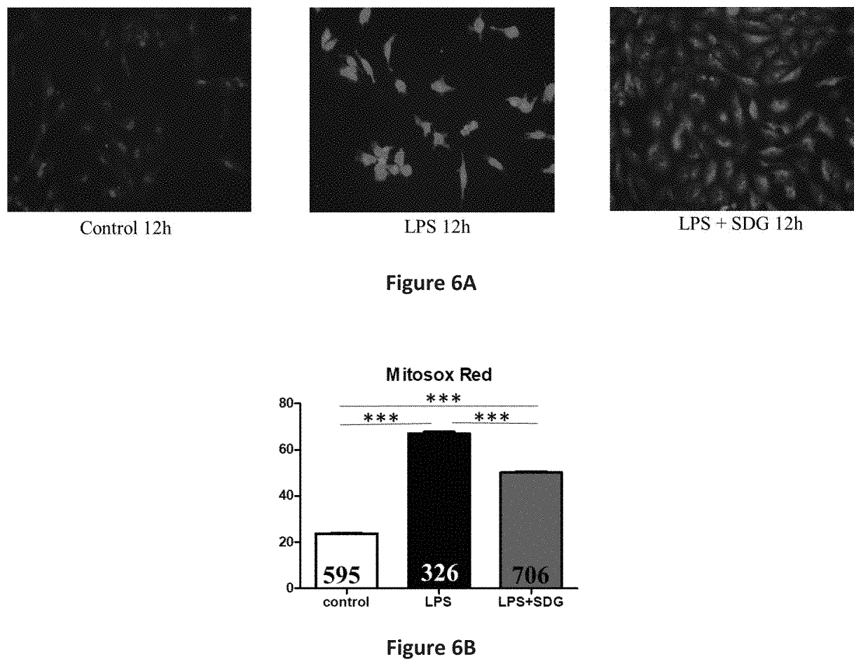

AC16 cells were either left untreated or treated for 12 hours with Liposaccharide (LPS) in the absence or presence of SDG, and subsequently stained with mitosox red. SDG treatment resulted in a statistically significant decrease in superoxide levels in LPS-treated cells (FIG. 6).

### Example 6

### SDG Suppresses the LPS-Mediated Increase in Mitochondrial Superoxide Generation in AC16 Cells

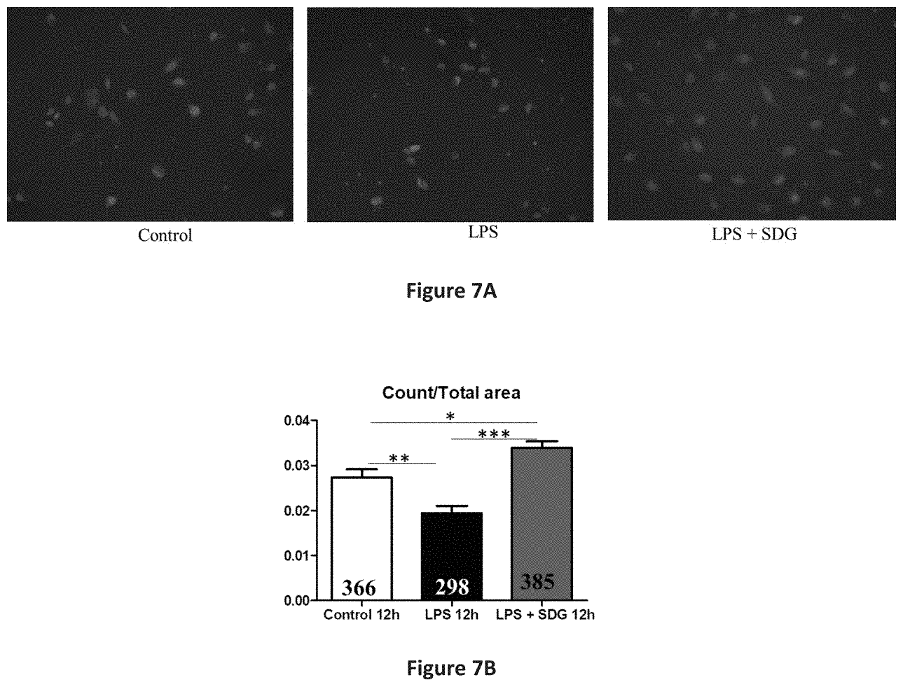

AC16 cells were either left untreated or treated for 12 hours with Liposaccharide (LPS) in the absence or presence of SDG, and mitochondria were stained. LPS treatment caused a statistically significant decrease in the number of mitochondria, but SDG treatment more than compensated for this LPS-dependent drop (FIG. 7).

### Example 7

### SDG Treatment Restores the Sepsis-Induced Changes in the mRNA Levels of Fusion and Fission Markers

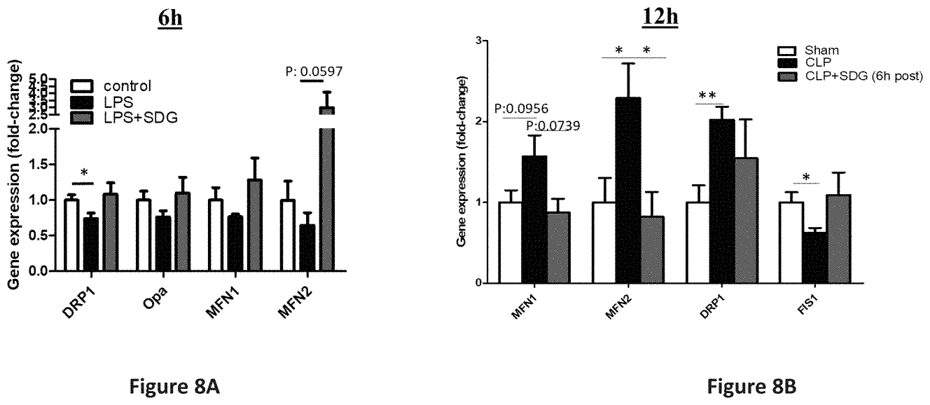

AC16 cells were either left untreated or treated with Liposaccharide (LPS) in the absence or presence of SDG; the levels of DRP1, Opa, MFN1, MFN2, and FIS1 mRNAs were measured in quantitative PCR. FIG. 8A shows that 6-hour LPS treatment caused a decrease in levels of all tested markers, although only the change in DRP1 levels was statistically significant. SDG treatment restored the levels of all the markers. FIG. 8B shows that 12 hour LPS treatment caused an increase in levels of all tested markers, wherein the change in DRP1, MFN2, and FIS1 levels was statistically significant. SDG treatment restored the levels of all the markers, although only the change in MFN2 levels was statistically significant.

### Example 8

### SDG Treatment of LPS-Stimulated AC16 Cells Increases the Expression of MCU and MICU1

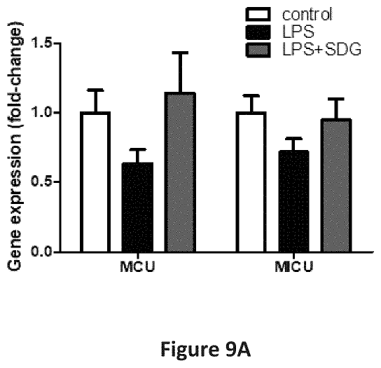

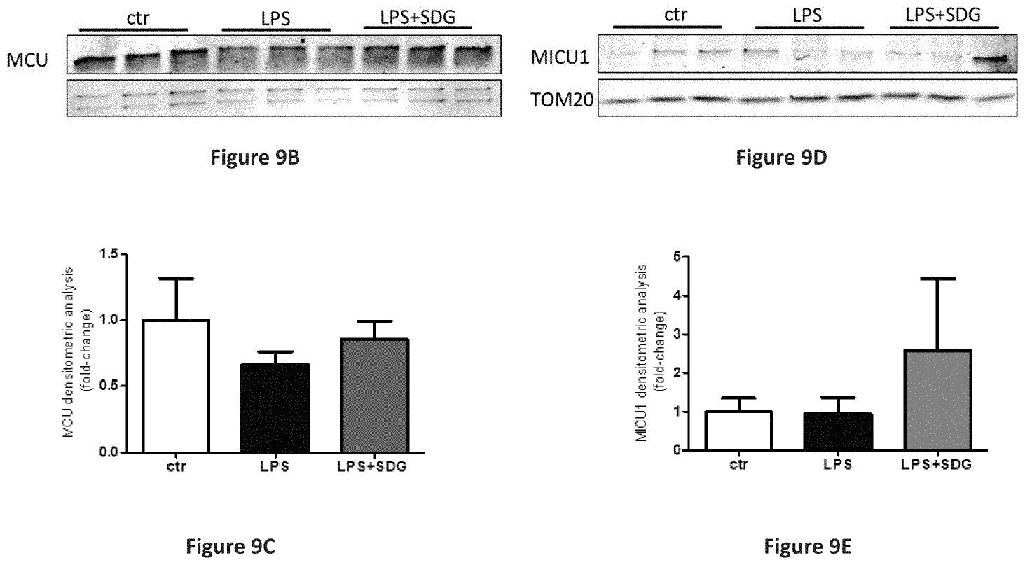

Mice were either sham treated or treated with LPS in with or without SDG (n=3 for each group) and the levels of MCU and MICU1 mRNAs were measured in quantitative PCR while the levels of protein were assessed through western blot. LPS suppressed levels of both MCU and MICU1 mRNA, but SDG treatment restored mRNA expression (FIG. 9A). FIG. 9B shows a western blot of MCU expression in each mouse. The graphical representation of the data averaged across each group (FIG. 9C) shows that SDG treatment reverses the LPS-mediated decrease of MCU protein level. FIG. 9D shows a western blot of MICU1 expression in each mouse. The graphical representation of the data averaged across each group (FIG. 9E) shows that SDG treatment increases MICU1 protein level.

### Example 9

### SDG Treatment of Septic Mice Increases the Protein Levels of MCU in the Heart Tissue

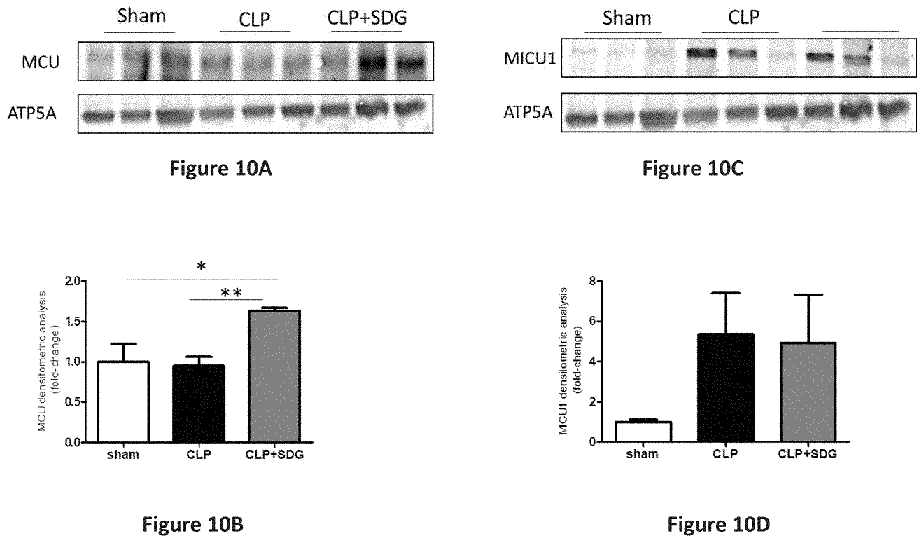

Mice were either sham treated (n=3) or subjected to cecal ligation puncture (CLP, n=6). SDG was administered to three mice following CLP. FIG. 10A shows a western blot of MCU expression in each mouse. The graphical representation of the data averaged across each group (FIG. 10B) shows that SDG treatment increases MCU protein level. FIG. 10C shows a western blot of MICU1 expression in each mouse. The graphical representation of the data averaged across each group (FIG. 10D) shows that CLP increases MICU1 protein level.

### Example 10

### SDG Increases the Oxygen Consumption Rate of Cardiomyocytes in Septic Mice

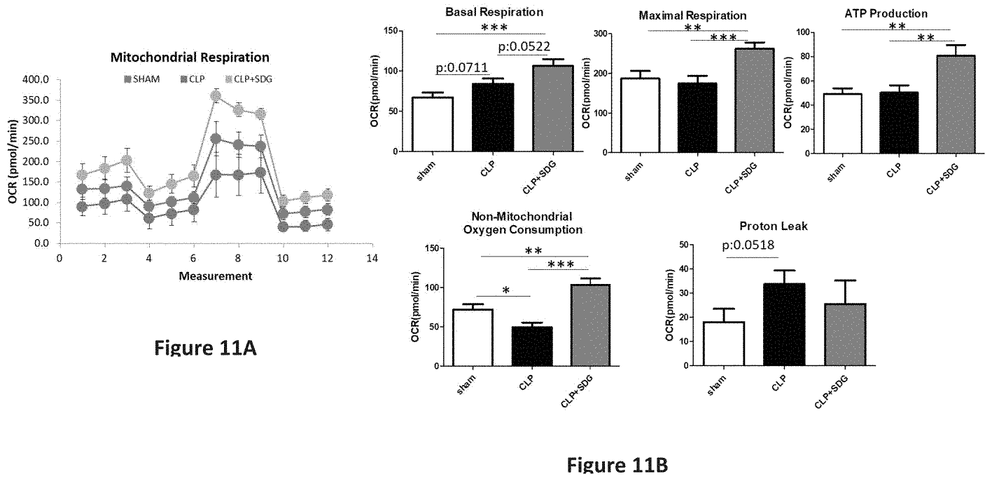

Mice were either sham treated or subjected to cecal ligation puncture followed by SDG treatment. The oxygen consumption rate (OCR) was measured in a seahorse assay. CLP resulted in decrease of all OCR measurements, while SDG treatment more than compensated for that decrease (FIG. 11A). CLP resulted in statistically significant decrease of non-mitochondrial oxygen consumption, while SDG treatment resulted in statistically significant increase in basal respiration, maximal respiration, ATP production, and non-mitochondrial oxygen consumption relative to the untreated levels (FIG. 11B). In addition, the SDG treatment resulted in statistically significant increase in maximal respiration, ATP production, and non-mitochondrial oxygen consumption relative to levels observed after CLP. Finally, no statistically significant changes in proton leakage were observed.

### Example 11

### The Antioxidant LGM2605 Improves Mitochondrial Function and Alleviates Septic Cardiomyopathy

Sepsis is characterized as the overwhelming immune response to infection ultimately leading to decreased tissue perfusion and organ damage. Myocardial dysfunction resulting from severe sepsis and septic shock is associated with high in hospital mortality approaching 50%. Evidence from our group has shown that energetic failure is a major component of myocardial dysfunction in sepsis, and that genetic and pharmacologic activation of metabolic pathways in cardiomyocytes improves cardiac function without resolving inflammation. Because ROS production is central to cellular metabolic health, we tested if the potent anti-oxidant synthetic lignan Secoisolariciresinol Diglucoside (SDG; LGM2605) would alleviate septic cardiac dysfunction in the cecal ligation and puncture (CLP)-based mouse model of peritonitis. We found that cardiac function measured by echocardiography was significantly impaired 12 hours post-surgery. Treatment of mice with SDG (100 mg/kg body weight, i.p.) 6 hours post-CLP surgery increased cardiac fractional shortening within 6 hours of SDG administration. To identify if the improvement in cardiac function was associated with changes in cardiac ROS levels, we stained cardiac tissue from these mice with dihydroethidium (DHE), which showed that SDG reduced ROS accumulation. This was consistent with in vitro studies, where we observed lower mitosox red staining in AC16 cardiomyocytes treated with combination of lipopolysaccharides (LPS) and SDG, as compared with cells treated with LPS alone. Seahorse XF analysis in primary cardiomyocytes obtained from adult C57BL/6 mice with CLP showed that SDG increased oxygen consumption rate (OCR) compared to CLP alone and sham operated mice, indicating increased mitochondrial respiration associated with ATP production. Aiming to identify the molecular pathway accounting for improved mitochondrial respiration, we assessed mitochondrial abundance using mitotracker staining and expression of mitochondrial calcium handling proteins. Treatment with SDG restored mitochondrial abundance in vitro and in vivo, and increased protein expression of the mitochondrial calcium uniporter, which regulates mitochondrial calcium uptake. Accordingly, mitochondrial calcium uptake was increased in isolated cardiomyocytes from these mice, which is known to improve mitochondrial function. Of note, SDG treatment did not restore the expression of fatty acid oxidation and glucose metabolism-related genes.

Taken together, our data show that SDG alleviates septic cardiac dysfunction via prevention of ROS accumulation, increased mitochondrial calcium uptake, and improved mitochondrial respiration. LGM2605 has a cardioprotective role in sepsis and can be used for therapies to manage sepsis.

Methods

Animal care, cecal ligation and puncture procedure and echocardiography—Animal protocols were approved by the Temple University Institutional Animal Care and Use Committee and were carried out in accordance with the NIH guidelines for the care and use of laboratory animals. Wild type (WT) 8-12 week old C57BL/6 mice were purchased from Jackson labs. Cecal ligation and puncture (CLP) was performed as previously described. Mice were anesthetized with 3.5% inhaled isofluorane. Under aseptic conditions, a 1 to 2 cm midline laparotomy was performed and exposure of the cecum with adjoining intestine. The cecum was tightly ligated at its base below the ileo-cecal valve at a distance of lcm and was punctured twice with a 19-gauge needle. The length of the ligated cecum was defined as the distance from the distal end of cecum to ligation point, which affects the degree of disease severity. Fecal material was extruded from the punctured cecum, and it was returned to the peritoneal cavity. The peritoneum and the skin were closed with three sutures. The mice were resuscitated by injecting subcutaneously lml of pre-warmed 0.9% saline solution to induce the hyperdynamic phase of sepsis and for post-operative analgesia the mice received subcutaneously buprenorphine (0.05 mg·kg body weight). Mice received a single intraperitoneal injection of LGM2605 (100 mg/kg i.p.) that was administrated either 2 h prior to CLP or 6 h post CLP. At 12 h post CLP, two-dimensional echocardiography was performed on anesthetized mice (1.5% inhaled isoflurane) using a VisualSonics Vevo 2100 machine. Echocardiographic images were recorded in a digital format. A single observer blinded to the respective treatments of mice analyzed short-axis m-mode images by LV trace. The number of mice used for each experiment are mentioned in the figure legends.

RNA purification and gene expression analysis—Total RNA was purified from AC16 cells or hearts using the TRIzol reagent according to the instructions of the manufacturer (Invitrogen). DNase-treated RNA was used for cDNA synthesis using the ProtoScript II First Strand cDNA Synthesis Kit (New England Biolabs). Quantitative real-time PCR was performed with the Sybr Select Master Mix (Applied Biosystems). Incorporation of the SYBR green dye into the PCR products was monitored with the Applied Biosystems StepOnePlus Real-Time PCR System. Samples were normalized against mouse 36B4 or human rps13 RNA.

Protein purification and analysis—Isolated heart tissue or AC16 cells were homogenized in radioimmune precipitation assay buffer containing protease and phosphatase inhibitors (Pierce Protease and Phosphatase Inhibitor Mini Tablets, Thermo Scientific). 30-50 μg of total protein extract was applied to SDS-PAGE and transferred onto nitrocellulose membranes.

Inflammatory cytokines measurement—Circulating levels of IL-1α, IL-1β, IL-6, IL-10 and TNFα were quantified simultaneously from frozen plasma samples using the Milliplex MAP Mouse Cytokine kit (MCYTOMAG-70K-05) following the kit specifications. Samples were read using the Luminex MAGPIX multiplexing unit.

Adult mouse cardiomyocytes isolation—Adult mouse cardiomyocytes (ACMs) were isolated from ventricles of C57BL/6 mice 12 hours post-sham surgery, CLP surgery or combined performance of CLP and treatment with LGM2605 at 6 hours post-surgery. Hearts from heparinized mice (90 USP; ip) were cannulated through the aorta. Hearts were perfused with perfusion buffer (120.4 mMNaCl, 14.7 mM KCL, 0.6 mM NaH2PO4, 0.6 mM KH2PO4, 1.2 mM MgSO4, 10 mM Hepes, 4.6 mM NaHCO₃, 30 mMtaurine, 10 mM BDM, 5.5 mM glucose; pH 7.4) for 3 min followed by digestion with perfusion buffer containing 19250 units Collagenese type II (Worthington), 5-6 mg trypsin and 0.02 mM CaCl2 for 7 min. Ventricles were gently teared into small pieces, perfusion buffer containing 5 mg/ml BSA and 0.125 mM CaCl2 was added and filtered with 100 μm nylon. The filtrate was pelleted by gravity for 5 min, centrifuged for 30 sec at 700 rpm and the pellet resuspended in perfusion buffer containing 5 mg/ml BSA and 0.225 mM CaCl2. The cells were pelleted by gravity for 10 min, centrifuged for 30 sec at 700 rpm and the pellet resuspended in perfusion buffer containing 5 mg/ml BSA and 0.525 mM CaCl2.

Cell culture—A human ventricular cardiomyocyte-derived cell line, designated AC-16, was used for some in vitro experiments. Cells were maintained in Dulbecco's modified Eagle's medium-nutrient mixture F-12 (DMEM-F-12; Invitrogen, Carlsbad, Calif.).

Measurement of mitochondrial superoxide, mitochondrial membrane potential and mitochondrial number—AC16 cells were cultured in sterile cell culture dishware at approximately 80% confluence. After overnight incubation, cells were treated with LPS (1 μg/ml), LPS and LGM2605 (50 μM). LPS and LGM2605 were diluted in serum free media and the control group was incubated as well with serum free media. At 12 hours after the treatment the AC16 cells were loaded with mitochondrial superoxide indicator MitoSOX Red (5 μM, M36008, Molecular Probes) and incubated for 30 minutes in the dark. Excess MitoSOX Red was removed following three washes with PBS solution. MitoSOX Red fluorescence was recorded at 510 (excitation) and 580 nm (emission).

To assess changes in the mitochondrial number, LPS stimulated AC16 cells and adult cardiomyocytes isolated 12 hours post-CLP were stained with 200 nM Mitotracker Red (M22425, Molecular Probes), and incubated for 30 minutes in the dark. Excess Mitotracker Red was removed with three washed of PBS solution. Mitotracker Red fluorescence was recorded at 581 (excitation) and 644 nm (emission).

To measure mitochondrial membrane potential, AC16 cells were stained with TMRM (62.5 nM, T668, Molecular Probes) for 30 minutes in the dark. TMRM stain was washed in warm PBS three times and imaged at 200× magnification using the Cy3 filter (510 nm excitation wavelength).

DHE staining of cardiac tissue—Live myocardium was isolated from mice 12 hours after surgery and sectioned into 10 sections using a clean razor blade. Tissue was stained with 20 μM dihydroethidium (DHE) for 30 minutes at room temperature and imaged on a Zeiss Axio Observer Z1 fluorescent microscope at 490±10 nm excitation and 632±30 nm emission. Oxidized DHE fluoresces red and intercalates DNA. Individual nuclei were measured within each visual field using Zeiss Zen Blue software, and visual fields were averaged to measure mean fluorescence intensity for each mouse.

Radioligand binding assay—Plasma membranes from excised mouse hearts were prepared, and saturation radio-ligand binding was performed as described previously, using ¹²⁵I-CYP (iodocyanopindolol; PerkinElmer, Waltham, Mass.) for β-AR density measurement. Data were analyzed by nonlinear regression analysis using GraphPad Prism (GraphPad Software, La Jolla, Calif.).

Seahorse analysis—Isolated primary ACMs were counted with Hematocytometer. Dead cells were detected with Trypan Blue Dye staining. Cells were platted (3000 cells per well) in XF96 Seahorse® plates pre-coated with laminin with 20 μg/ml laminin (Invitrogen, 23017). In order to assess oxygen consumption rates (OCR) for fatty acid oxidation (FAO) recordings, cells were incubated in substrate limited medium (DMEM containing 10 mM Glucose, 1.025 mM CaCl2, 0.5 mM carnitine, pH=7.4) and assayed with fatty acid oxidation medium as per manufacturer's protocol. Before starting the assay, 1 mM palmitate conjugated with BSA was added in each well. Drugs used for maximal response during fatty acid oxidation were: Oligomycin (3 μM) (Sigma, 04875) which blocks complex V, FCCP (2 μM) (Sigma, C2920) that leads to the collapse of the proton gradient, and Rotenone/Antimycin A (0.5 μM) (Sigma, A8674)/(Sigma, R8875) where rotenone blocks complex I and antimycin A blocks complex III. The pre-hydrated with XF assay calibrant, XF cartridges were filled with the drugs and the cartridge was calibrated for 30 minutes in Seahorse Analyzer. All experiments were performed at 37° C. Calculations were made as described in the Seahorse manual and XF Seahorse Mito Stress Test kit user guide. Briefly, basal respiration was calculated with subtraction of non-mitochondrial respiration rate from the last measurement prior to first injection. Maximal respiration was calculated by subtraction of the non-mitochondrial respiration measurement from maximum measurement after FCCP injection. ATP synthesis-related OCR was obtained indirectly by measuring ATP-linked respiration in the presence of complex V inhibitor (Oligomycin). The decrease of oxygen consumption rate representing the portion of basal respiration that was used to drive ATP synthesis was calculated with subtraction of the minimum measurement after Oligomycin injection from the last measurement prior to Oligomycin injection. Spare Respiratory Capacity was equal to (maximum respiration)−(basal respiration).

In vivo cardiac adrenergic sensitivity measurements—Hemodynamics measurements were performed, as published²¹. Mice were anesthetized with 2% Tribromoethanol (Avertin). The right carotid artery was cannulated with a 1.4 French micro-manometer (Millar Instruments, Houston, Tex.) and was advanced into LV cavity, measuring LV pressure, LV end-diastolic pressure (LVEDP) and heart rate (HR). These parameters as well as maximal values of the instantaneous first derivative of LV pressure (+dP/dtmax, as a measure of cardiac contractility) and minimum values of the instantaneous first derivative of LV pressure (−dP/dtmin, as a measure of cardiac relaxation) were recorded at baseline and after administration, through the jugular vein, of increasing doses of the β-adrenergic receptor (βAR) agonist, isoproterenol (0.1 ng, 0.5 ng, 1 ng, 5 ng, 10 ng). Data was recorded and analyzed on a PowerLab System (AD Instruments).

Calcium uptake assay in permeabilized adult cardiomyocytes Cardiomyocytes were isolated from mice 12 hours after CLP or sham surgery and 6 hours after LGM2605 or saline injection. Calcium uptake was performed as previously described²² and detailed below. Before permeabilization, cardiomyocytes were washed in a Ca free buffer (120 mM NaCl, 5 mM KCL 1 mM KH₂PO₄, 0.2 mM MgCl₂, 0.1 mM EGTA and 20 mM HEPES-NaOH, at pH 7.4) and stored on ice for at least 10 min. Cardiomyocytes were pelleted by centrifugation and transferred to an intracellular-like medium (permeabilization buffer: 120 mM KCl, 10 mM. NaCl, 1 mM KH₂PO₄, 20 mM, 4 HEPES-Tris, at pH 7.2, protease inhibitors (EDTA-free complete tablets, Roche Applied Science), 2 μM thapsigargin and digitonin (40 μg ml⁻¹)). The cell suspension supplemented with succinate (2 mM) was placed in a fluorimeter and permeabilized by gentle stirring. Fura2FF (0.5 μM) was added at 0 s, and JC-1 (800 nM) at 20 s. Fluorescence signal was monitored in a temperature-controlled (37° C.) multiwavelength-excitation dual-wavelength-emission spectrofluorometer (Delta RAM, Photon Technology International) using 490-nm excitation/535-nm emission for the JC-1 monomer. 570-nm excitation/595-nm emission for the J-aggregate of JC-1 and 340-nm/380-nm for Fura2FF. At 400 s, a single 10 μM Ca²⁺ pulse was added, and changes in cytosolic [Ca²] was monitored. CCCP was added at 750 s to collapse the mitochondrial membrane potential and measure calcium expelled from the mitochondria.

Statistical analysis—Results are presented as mean±SEM. The unpaired t-test was used for comparisons of two means; a 2-tailed value of P<0.05 was considered statistically significant. For groups of 2 or more ANOVA was used with Bonferroni post-hoc test (Prism v5, GraphPad Software).

Results

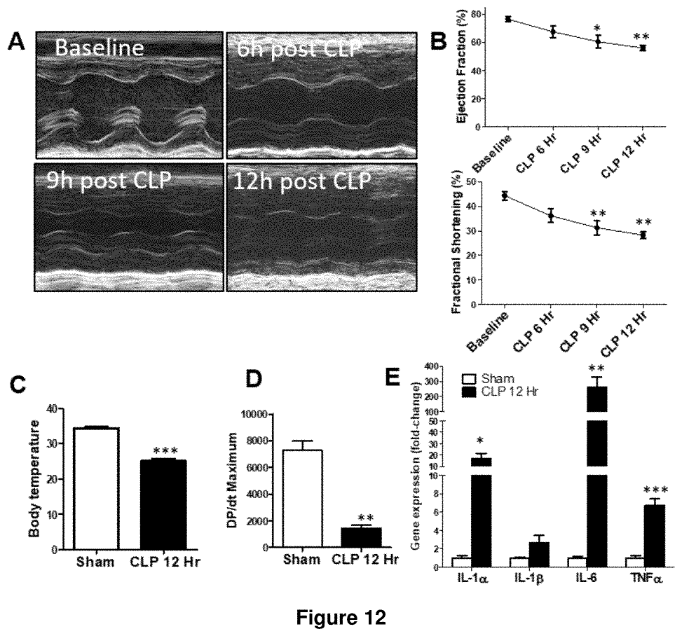

LGM2605 prevents cardiac dysfunction in a mouse model of sepsis induced by cecal ligation and puncture (CLP)—We induced mid-to-low grade sepsis (ligation site: lcm) in male C57BL/6 mice using CLP and assessed cardiac function with 2D-echo up to 12 h post-surgery. Cardiac function begins declining 6 h post-CLP and septic mice demonstrated significant cardiac dysfunction 9 h post-CLP, which deteriorated further 12 h post-CLP (FIG. 12A-B). Septic mice showed significant decreases in body temperature (FIG. 12C), as well as in contractility represented by dP/dt^{max} (FIG. 12D) and increased expression of cardiac inflammatory genes (FIG. 12E) 12 h post-CLP.

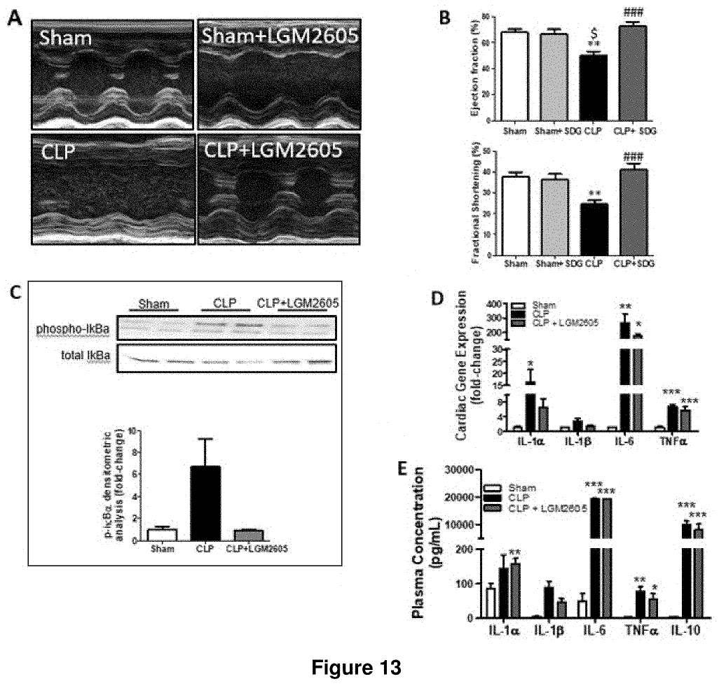

To assess the effect of LGM2605 in septic cardiac dysfunction, we first administered LGM2605 via intraperitoneal injection (100 mg/kg body weight) 2 h prior to CLP in one group of mice and 6 h post-CLP in another group of mice. Both pre-CLP (Suppl. FIG. 12A-B and Table 1) and post-CLP (FIG. 13A-B and Table 1) treatments prevented CLP-mediated cardiac dysfunction. As opposed to septic mice that did not receive LGM2605, which showed a 17.82% reduction in ejection fraction, and 12.06% reduction in fractional shortening, the mice that were treated with LGM2605 had normal systolic function. Thus, LGM2605 improves cardiac function in sepsis, when administered either preventively or after CLP surgery.

LGM2605 influences cardiac NF-kB activation but not cardiac expression and plasma inflammatory cytokines levels—To assess if the cardioprotective effect of LGM2605 relies on anti-inflammatory properties, we tested the expression of cardiac inflammatory markers in the hearts of septic C57BL/6 mice. Mice with CLP had increased phosphorylation of IκBα, suggesting increased NF-κB activation, which was prevented by LGM2605 (FIG. 13C). Analysis of inflammatory markers in septic mice showed that LGM2605 did not reduce mRNA levels of IL-la, IL-1β, IL-6, and TNFα at 6 hours (FIG. 13A) or 12 hours post-CLP (FIG. 13D). Accordingly, LGM2605 administration did not reduce circulating levels of pro-inflammatory cytokines IL-la, IL-1β, IL-6, and TNFα, or the anti-inflammatory cytokine IL-10, which is also elevated during sepsis (FIG. 13E). Thus, although NFκB signaling is alleviated by LGM2605 treatment, LGM2605-mediated cardiac function improvement does not lower production of inflammatory cytokines.

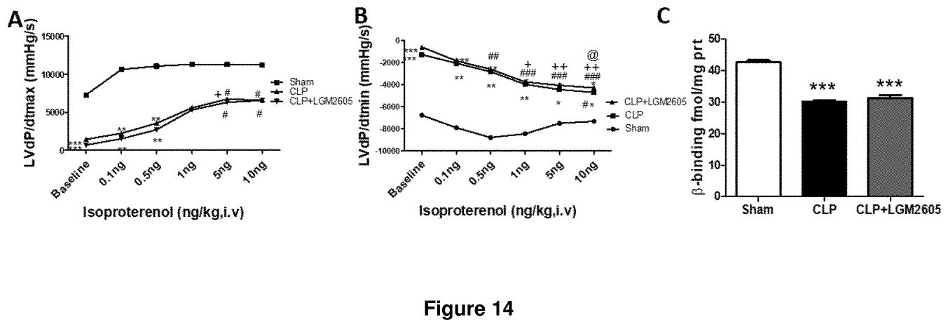

LGM2605-mediated improvement in cardiac function is not associated with altered β-AR signaling—As sepsis affects cardiac contractility, which is mainly controlled by (3-adrenergic receptor (β-AR) signaling, we tested if LGM2605 improves cardiac function by improving β-AR sensitivity. Hemodynamic measurements showed that septic mice had lower basal myocardial LVdP/dtₘₐₓ (FIG. 14B) and LVdP/dtₘᵢₙ (FIG. 14C) with or without LGM2605 administration, compared to sham surgery. Responsiveness to isoproterenol was less robust in both septic groups regardless of LGM2605 treatment (FIG. 14A, 14B). As basal levels of LVdP/dtₘₐₓ and LVdP/dtₘᵢₙ were lower in mice with CLP, we performed radioligand binding assay to assess the density of β-AR in hearts obtained from septic mice (12 h post-CLP) that were treated with LGM2605 (6 h post-CLP). This analysis showed a significant decrease of cardiac β-AR density in mice that underwent CLP, which was not reversed by treatment with LGM2605 (FIG. 14C).

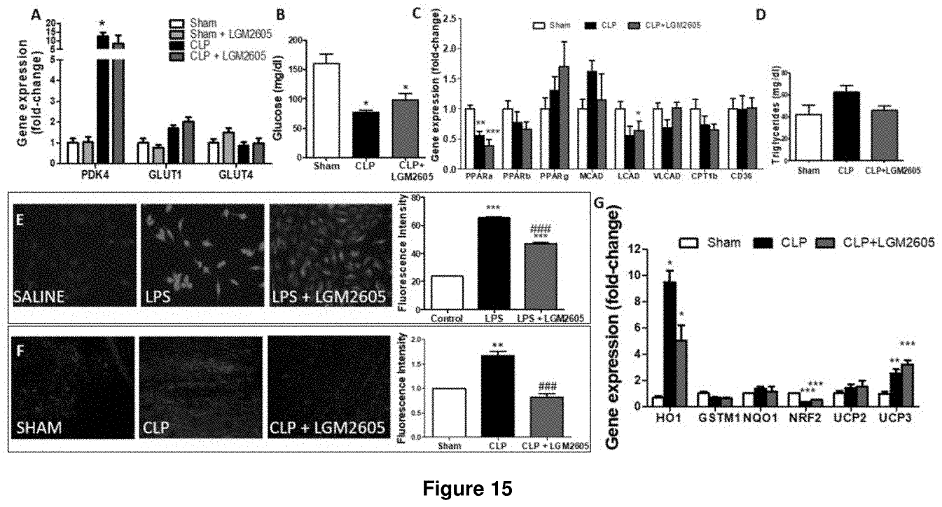

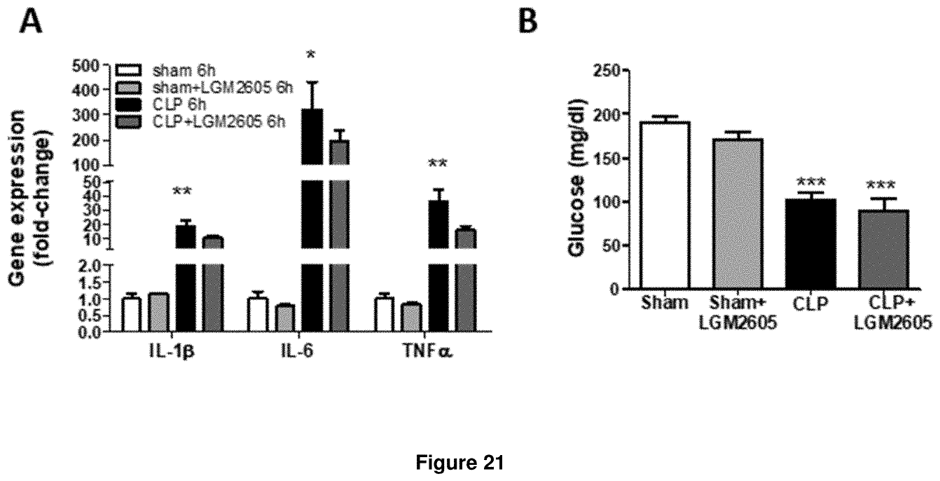

LGM2605 does not affect glucose and fatty-acid metabolism related gene expression—We measured cardiac mRNA levels of glucose uptake and catabolism markers including GLUT1, GLUT4 and PDK4 in mice that underwent CLP with and without LGM2605. GLUT1 and GLUT4 cardiac mRNA levels did not change significantly in septic heart tissue, but we observed a significant increase in the mRNA levels of cardiac PDK4, which inhibits pyruvate utilization by inactivating pyruvate dehydrogenase (FIG. 15A). This increase was not alleviated by administration of LGM2605, suggesting that LGM2605 does not prevent sepsis-associated reduction in glucose utilization (FIG. 15A). We measured plasma glucose levels in septic mice 6 hours (FIG. 21B) and 12 hours (FIG. 15B) post-CLP and observed that hypoglycemia in septic mice was not alleviated by the administration of LGM2605.

We then assessed cardiac expression of genes associated with fatty-acid metabolism, which constitutes approximately 70% of the cardiac ATP production²³, that are known to be affected during sepsis. Mice with CLP had lower expression levels of PPARα and LCAD, while PPARβ/δ, PPARγ, MCAD, VLCAD, CPT1β and CD36 mRNA levels were not significantly altered (FIG. 15C). LGM2605 did not reverse the CLP-mediated changes in the PPARα and LCAD (FIG. 15C). Plasma triglyceride levels showed a trend of increase, which did not occur in septic mice treated with LGM2605 (FIG. 15D). Collectively, these data suggest that the beneficial effect of LGM2605 in septic cardiac dysfunction is not associated with alterations in fatty-acid metabolism-related gene expression.

LGM2605 alleviates oxidative stress without altering antioxidant-related gene expression—We measured mitochondrial superoxide generation using Mitosox Red staining in AC16 cells treated with E. coli lipopolysaccharides (LPS). Treatment of AC16 cells with LPS for 12 h increased mitochondrial superoxide levels, which was suppressed by LGM2605 (FIG. 15E). Accordingly, dihydroethidium (DHE) staining of ventricular tissue isolated from septic mice 12 hours post-CLP showed increased staining intensity which was alleviated significantly by LGM2605 administration (FIG. 15F). The beneficial effect of LGM2605 was not accompanied by prevention of the CLP-mediated changes in cardiac expression of antioxidant genes, including nuclear respiratory factor 2 (NRF2), heme oxygenase 1 (HO1), glutathione S-transferase Mu 1 (GSTM1), NAD(P)H:quinone oxidoreductase 1 (NQO1), and uncoupling proteins 2 and 3 (UCP2, UCP3) (FIG. 15G). Taken together, these results show that the beneficial effect of LGM2605 relies on direct alleviation of ROS accumulation and not on transcriptional effects on the gene expression program of antioxidant systems.

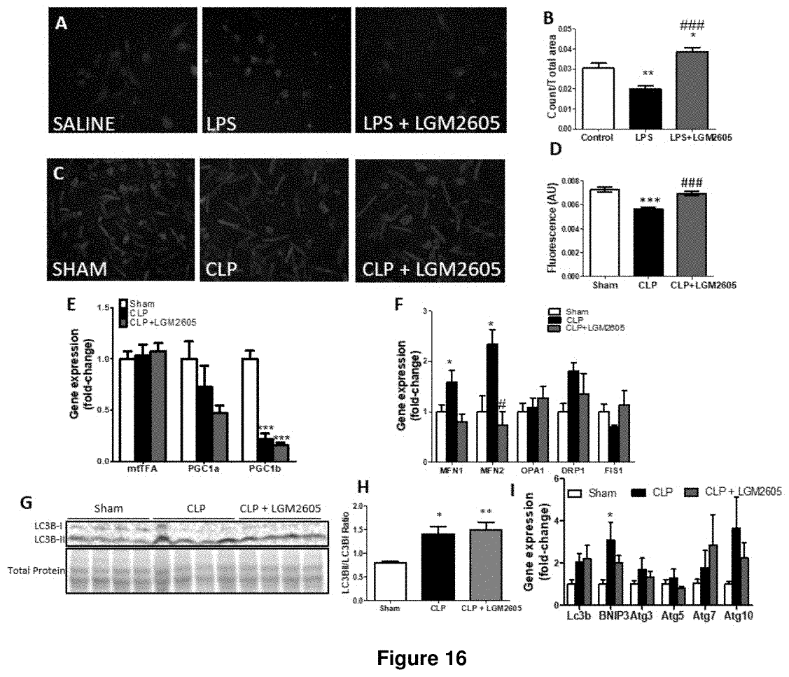

LGM2605 increases mitochondrial abundance without affecting mitochondrial biogenesis-related gene expression or autophagy markers—To assess whether the beneficial effect of LGM2605 involves changes in mitochondrial number that is known to be affected in septic cardiac dysfunction, we treated AC16 cells with LPS and LGM2605 for 12 h and stained them with Mitotracker Red. LPS treatment decreased mitochondrial number, which was prevented by treatment with LGM2605 (FIG. 16A-B). In accordance with these results, Mitotracker Staining analysis performed in adult cardiomyocytes isolated from septic mice 12 hours post-CLP suggests that LGM2605 prevents CLP-associated reduction in mitochondrial abundance (FIG. 16C-D).

To investigate the mechanism that may mediate LGM2605-driven restoration of mitochondria abundance, we measured the expression of various markers of mitochondrial biogenesis, fusion/fission, and mitophagy. Cardiac gene expression of mitochondrial biogenesis markers including PGC1α and PGC1β were reduced 12 hours post-CLP, whereas mtTFA was not significantly changed. None of these changes was affected by LGM2605 (FIG. 16E). We then examined whether the effect of LGM2605 was associated with changes in the expression of mitochondrial fusion and fission markers. Heart tissue of mice that underwent CLP had increased mRNA levels of MFN1, MFN2, and DRP1, and a trend toward reduced mRNA levels of Fis1 (FIG. 16F). LGM2605 treatment restored the CLP-mediated changes in MFN1, MFN2, DRP1, and FIS1 expression (FIG. 16F), suggesting that LGM2605 may affect the dynamics between mitochondrial fission and fusion by suppressing expression of CLP-induced fusion markers.

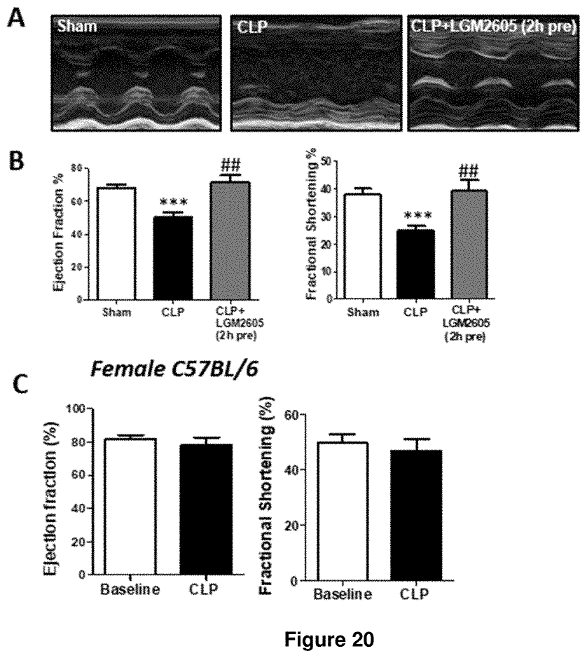

To assess activation of autophagy pathways that mediate mitochondrial flux in sepsis, we measured conversion of LC3BI to LC3BII, a known marker of autophagosome formation. We found increased LC3BII/LC3BI ratio (FIG. 16G, 5H) demonstrating activation of autophagy pathways in cardiac tissue isolated from septic mice. We further found an induction of Map11c3b and BNIP3 mRNA levels (FIG. 20). Neither marker was alleviated by LGM2605 suggesting that LGM2605 does not prevent activation of autophagy pathways.

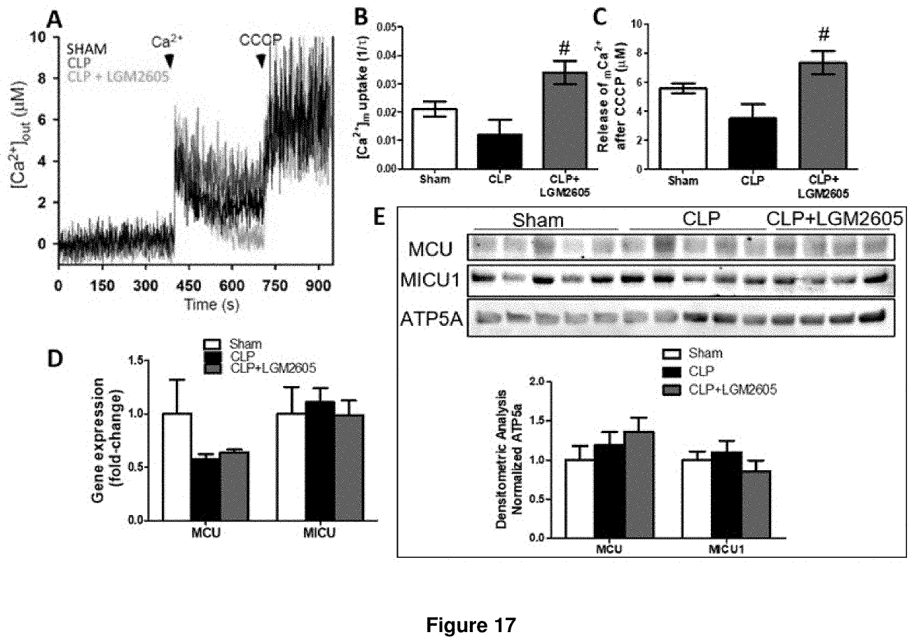

Increased cardiac mitochondria abundance in LGM2605-treated mice is associated with increased mitochondrial calcium uptake and oxygen consumption rate—Mitochondrial calcium uptake was assessed in digitonin-permeabilized adult cardiomyocytes treated with thapsigargin that inhibits SERCA-mediated Ca²⁺ uptake by the endoplasmic reticulum (FIG. 17A). Cardiomyocytes isolated from mice 12 h after CLP surgery showed reduced Ca²⁺ uptake and lower release of Ca²⁺ following CCCP administration (FIG. 17B,17C). We found that administration of LGM2605 to septic mice increased mitochondrial Ca²⁺ uptake rate and release of Ca²⁺ following CCCP administration beyond that observed for sham operated mice (FIG. 17B, 17C). To determine if changes in calcium uptake were associated with changes in mitochondrial calcium uptake associated genes, we measured expression of mitochondrial calcium uniporter (MCU) and mitochondrial calcium uptake protein (MICU)1. On the mRNA and protein level, neither MCU or MICU1 were altered 12 hours post-CLP, and LGM2605 did not affect expression of these proteins. (FIG. 17D-E).

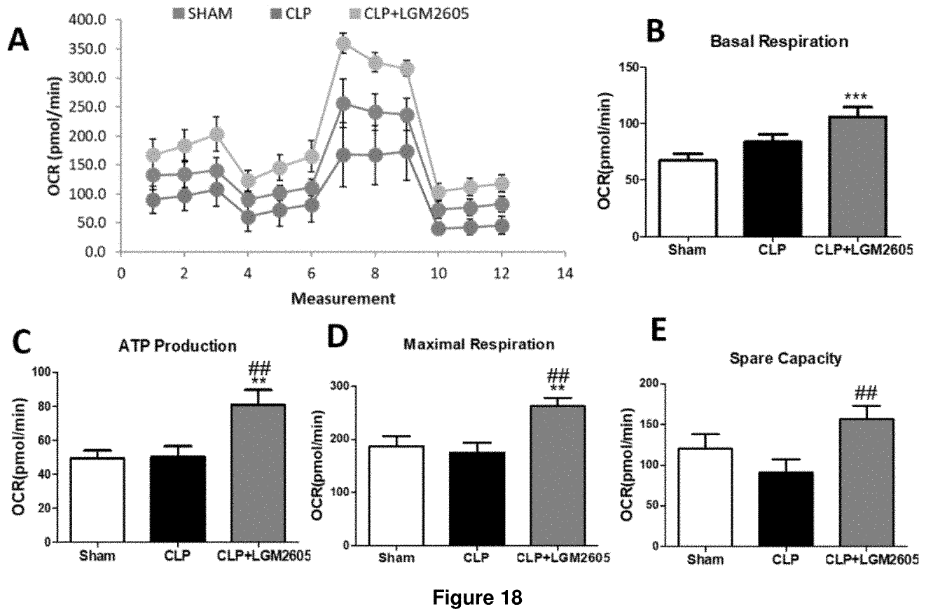

Increased mitochondrial calcium uptake was accompanied by increased oxygen consumption rate, as measured with Seahorse XF analysis of adult cardiomyocytes isolated from septic mice that were treated with LGM2605 (FIG. 18A-E). Administration of LGM2605 in mice, which had undergone CLP, increased basal respiration levels (FIG. 18B) and oxygen consumption rate associated with ATP synthesis (FIG. 18C), compared to both CLP and sham mice. Maximal respiration and respiratory spare capacity were also markedly elevated by LGM2605 treatment (FIG. 18D-E), suggesting that LGM2605 may increase the ability of mitochondria to meet increased energetic demand during cardiac stress.

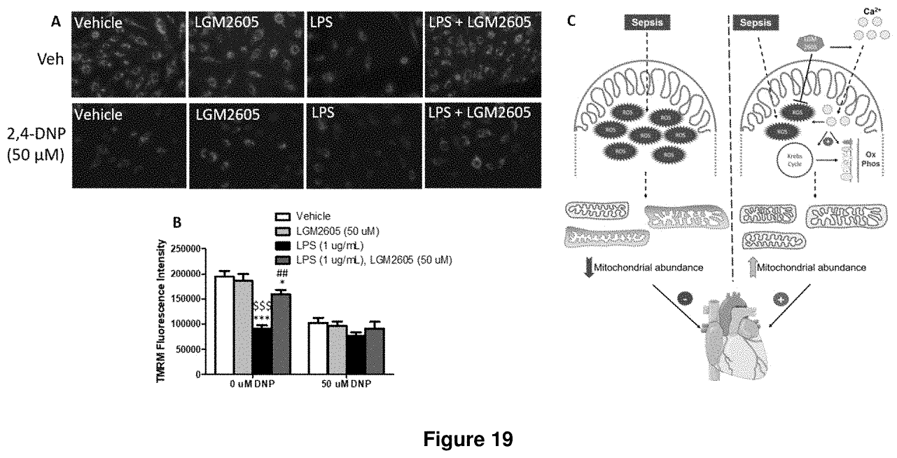

LGM2605 prevents mitochondrial membrane depolarization—Because the mitochondrial membrane potential is the major driving force for mitochondrial calcium uptake and mitochondrial respiration, we tested if LGM2605 alters mitochondrial depolarization using TMRM staining, which is sequestered by active mitochondria dependent on the mitochondrial membrane potential. LPS treatment significantly reduced TMRM staining intensity compared to vehicle and LGM2605 treated controls, suggesting that LPS induces a reduction in the mitochondrial membrane potential (FIG. 19A, 19B). Treatment with LGM2605 significantly restored TMRM staining toward baseline (FIG. 19A, 19B).

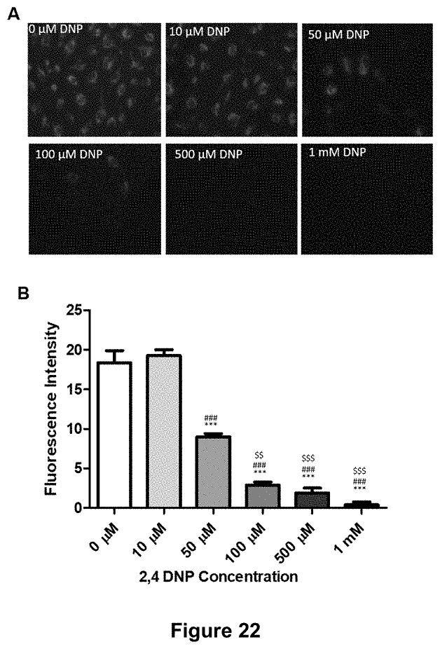

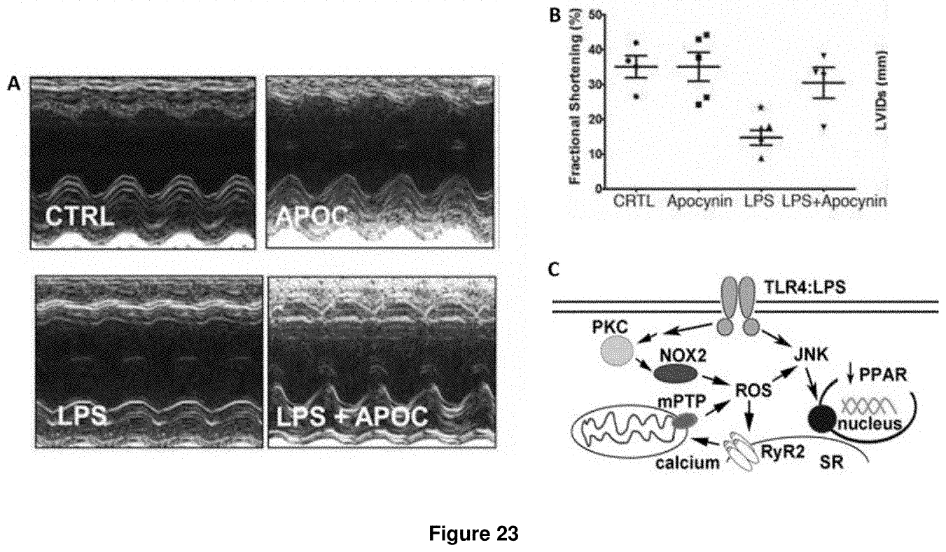

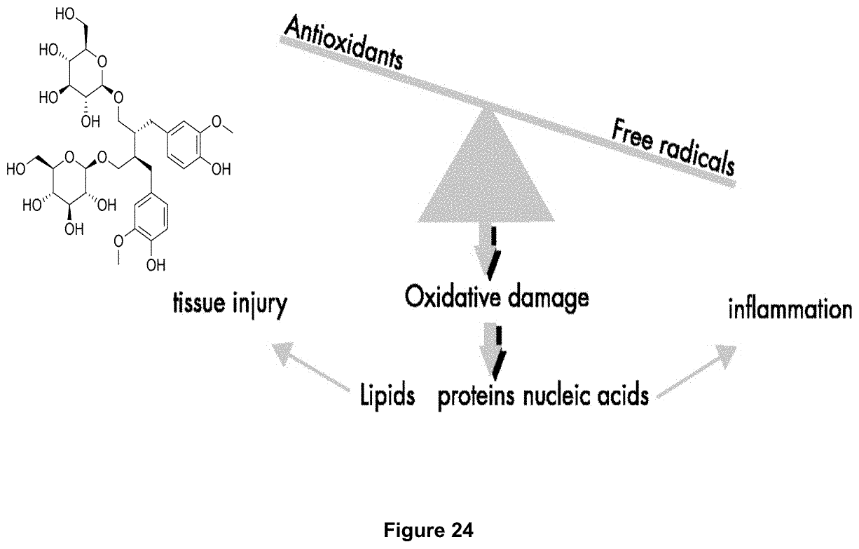

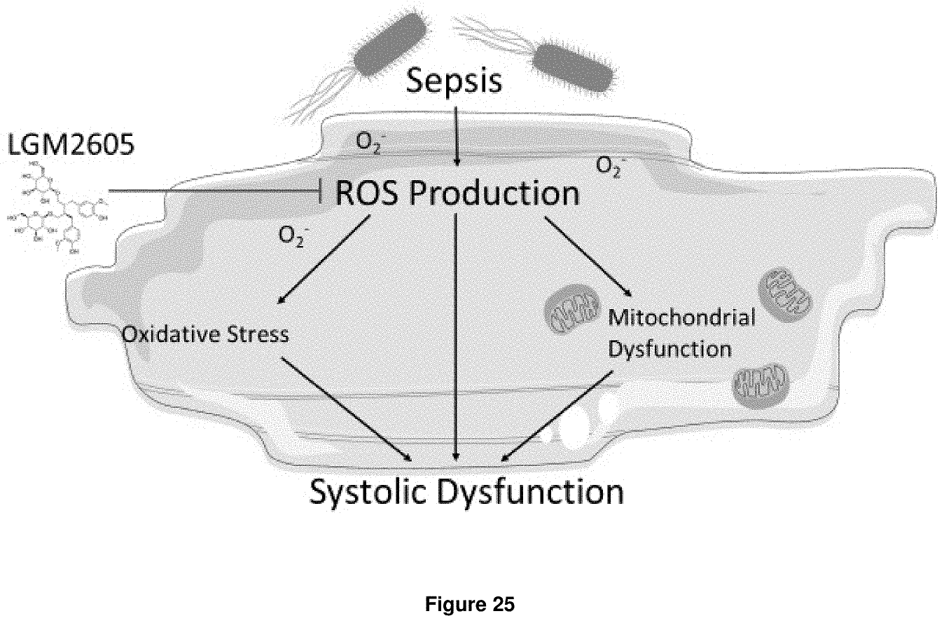

As a control experiment, we tested if LGM2605 would affect membrane depolarization that is driven by the uncoupling agent 2,4-dinitrophenol (2,4-DNP), which depolarizes the mitochondrial membrane independent of ROS generation. First, to select the right dose of 2,4-DNP that would not incur significant toxicity for AC16 cells, we applied a series of treatments with increasing concentrations of 2,4-DNP (FIG. 22). Based on this analysis, we selected to treat cells with 50 μM 2,4-DNP. Combined treatment of AC16 cells with LGM2605 and 2,4-DNP showed that LGM2605 lost its beneficial effect in restoring TMRM staining in cells that received 50 μM 2,4-DNP either with or without LPS (FIG. 19B).

TABLE 1
Average short-axis echocardiography-derived measurements and sample sizes for mice included in the study.

| Treatment | | | | LV | LV Mass | LV | LV | | | | | | |
|---|---|---|---|---|---|---|---|---|---|---|---|---|---|
| Group | n | EF | FS | Mass | (Corrected) | Vol; d | Vol; s | IVS; d | IVS; s | LVID; d | LVID; s | LVPW; d | LVPW; s |
| Sham | 9 | 67.9 | 37.8 | 130.5 | 104.4 | 76.9 | 25.2 | 0.8 | 1.4 | 4.1 | 2.6 | 0.8 | 1.2 |
| Sham + LGM2605 | 4 | 66.7 | 36.4 | 146.7 | 117.4 | 55.4 | 17.9 | 1.1 | 1.5 | 3.6 | 2.3 | 1.0 | 1.4 |
| (6 hr) | | | | | | | | | | | | | |
| CLP | 12 | 50.3 | 24.8 | 119.5 | 95.6 | 32.6 | 16.4 | 1.3 | 1.6 | 2.9 | 2.2 | 1.0 | 1.3 |
| CLP + LGM2605 | 4 | 71.7 | 39.3 | 97.2 | 77.8 | 18.6 | 5.3 | 1.3 | 1.6 | 2.3 | 1.4 | 1.1 | 1.5 |
| (−2 hr) | | | | | | | | | | | | | |
| CLP + LGM2605 | 13 | 72.7 | 41.3 | 129.2 | 103.3 | 30.2 | 8.4 | 1.4 | 1.8 | 2.8 | 1.6 | 1.1 | 1.6 |
| (6 hr) | | | | | | | | | | | | | |

Sepsis is the most common cause of death among critically ill patients in intensive care units (ICU); particularly when it is accompanied by acute organ dysfunction. One hospital-based study found that 43% of patients with bacteremia had increased serum troponin, indicative of myocardial damage. Other clinical studies have shown that the presence of cardiovascular dysfunction in sepsis is associated with significantly increased mortality rate of 70% to 90% compared with 20% mortality in septic patients without cardiovascular impairment. Despite years of research, the pathophysiology of sepsis-induced myocardial dysfunction has not yet been defined, and the responsible cellular mechanisms still remain unclear. No effective treatments or specific medications are used in clinical practice to reverse sepsis-induced cardiomyopathy. The pathophysiology of septic cardiac dysfunction has been attributed to increased oxidative stress, elevated inflammation, impaired β-adrenergic signaling, activation of apoptosis, suppression of metabolic pathways, and reduced ATP synthesis in the cardiomyocytes.

Previous evidence from our group has shown that a major component of myocardial dysfunction in sepsis is energetic failure, the correction of which improves cardiac function despite increased levels of inflammatory cytokines. Mitochondria constitute major cellular organelles involved in the energetic machinery of the heart and other organs. Thus, mitochondrial dysfunction can be detrimental for cardiac function in sepsis and other diseases. ROS such as superoxide and peroxide compromise mitochondrial integrity and function during sepsis. Previous work from our team has shown that the inhibition of NOX2, which is an extramitochondrial protein involved in the generation of superoxide, alleviated oxidative stress and preserved cardiac function in a murine model of sepsis, indicating a crucial role of ROS stress in aberrant cardiac function associated with sepsis.

In the present study, we focused on the effects of antioxidant therapy in mitochondrial function during sepsis. The antioxidant LGM2605 successfully restored normal cardiac function in septic mice when administered either after or prior to the induction of sepsis. The cardioprotective effect of LGM2605 was associated with restoration in mitochondrial abundance, and a reduction in LPS and CLP-mediated increase in ROS in vitro and in vivo respectively. Mitochondria are central to the detrimental effects of oxidative stress on cellular function in cardiomyocytes. Increased ROS generation is associated with mitochondrial membrane depolarization, reduced mitochondrial respiration, and initiation of apoptotic pathways³³. In our study, we observed an increase in cardiomyocyte mitochondrial respiration and preservation of mitochondrial membrane potential with LGM2605 antioxidant therapy, suggesting that LGM2605 protects mitochondrial function.

Alterations in mitochondrial abundance, regulation of fission/fusion pathways, and energetic failure have been described in cardiac disease. Our present study indicates the role of reduced mitochondrial abundance resulting from oxidative stress in aggravating cardiac function in sepsis. LGM2605 treatment was accompanied by increased mitochondrial calcium uptake and mitochondrial respiration. Calcium import in mitochondria takes place primarily through the mitochondria calcium uniporter (MCU) and is driven by the mitochondrial membrane potential. As MCU protein levels are not significantly altered in septic mice treated with LGM2605, the improvement in mitochondrial calcium uptake may be driven by restoration of mitochondrial potential that the beneficial treatment incurs and the increase in mitochondrial abundance. Mitochondrial calcium serves as a regulator of enzymes associated with fatty acid and pyruvate oxidation, the Krebs cycle and oxidative phosphorylation and stimulates enzymatic activity associated with cellular respiration. On the other hand, inhibition of increased Ca²⁺ uptake has been proposed as a therapeutic intervention during cardiac stress, and unregulated Ca²⁺ uptake by mitochondria increases ROS production. In our study, increased Ca²⁺ uptake was associated with lower ROS accumulation. This effect may be attributed to the anti-oxidant effect of LGM2605, which seems to act as a dual mitochondrial Ca²⁺ uptake inducer and ROS scavenger (FIG. 19C).

Increased mitochondrial calcium uptake has also been proposed as an essential process underlying the energetic adaptations to adrenergic signaling in the heart. A study, using a different sepsis animal model, reported reduced mitochondrial calcium uptake in isolated non-permeabilized rat cardiomyocytes. The study indicated that reduced calcium uptake may underlie lower responsiveness to adrenergic challenge in septic rats. In our study, LGM2605 increased calcium uptake in permeabilized cardiomyocytes but did not improve β-AR responsiveness. Therefore, increased mitochondrial abundance and calcium uptake can restore mitochondrial respiration but they do not suffice to reverse lack of β-AR responsiveness in sepsis.

Based on our previous studies that identified energetic failure as a major cause of septic cardiomyopathy, our present study focuses on the role of the oxidative stress-mediated impairment of mitochondrial parameters in aggravating cardiac function in sepsis. We observed that sepsis activated the expression of mitofusin genes, which was suppressed by LGM2605. This result show that LGM2605 alleviates cardiac stress thereby reducing stress-induced mitochondrial hyperfusion (such hyperfusion has been previously described as a pre-apoptotic cellular response to stress conditions). Others have shown that oxidative stress promotes mitochondrial hyperfusion in myocytes. Surprisingly, LGM2605 did not affect gene expression associated with mitochondrial biogenesis or activation of autophagy pathways, which mediate mitophagy. Importantly, our gene expression analyses show trends of restored expression of autophagy-related markers by LGM2605. This response may constitute early secondary signals that ameliorate mitophagy due to lower oxidative stress, preserved mitochondrial membrane potential, increased mitochondrial respiration, and reduced mitochondrial damage. In support of this, our results show a trend for reduced expression of the mitochondrial damage marker BNIP3 following LGM2605 administration, indicating that the mitochondrial-protective properties of LGM2605 can prevent mitochondrial damage.

To conclude, our study demonstrated the role of the anti-oxidant LGM2605, a chemically synthesized SDG, in the prevention of septic cardiac dysfunction in a mouse model of polymicrobial sepsis. The beneficial effect of LGM2605 was associated with reduced oxidative stress, preserved mitochondrial membrane potential, and increased mitochondria abundance and respiration. Thus, LGM2605 can be used as a therapeutic agent for septic cardiomyopathy.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

1. A method for treating or preventing sepsis-induced cardiomyopathy in a subject in need thereof, comprising: administering to said subject an effective amount of secoisolariciresinol diglucoside (SDG), an analog thereof, a stereoisomer thereof, or a combination thereof, thereby treating sepsis-induced cardiomyopathy in said subject.

2. The method of claim 1, wherein said SDG is (S,S)-SDG or (R,R)-SDG.

3. (canceled)

4. The method of claim 1, wherein said SDG is a synthetic SDG.

5. (canceled)

6. The method of claim 1, wherein said step of administering is performed orally or is performed intravenously.

7. (canceled)

8. (canceled)

9. The method of claim 1, wherein the subject is a human subject.

10. The method of claim 1, wherein said sepsis is associated with systemic inflammatory response syndrome (SIRS), infection, or multiple organ dysfunction syndrome.

11. The method of claim 1, wherein said sepsis is associated with a trauma or an injury or said sepsis is associated with a medical treatment.

12. (canceled)

13. (canceled)

14. (canceled)

15. A method for maintaining cardiac function and/or for improving cardiac contractility and/or cardiomyocyte mitochondrial function in a subject having sepsis, comprising: administering to said subject an effective amount of secoisolariciresinol diglucoside (SDG), an analog thereof, a stereoisomer thereof, or a combination thereof.

16. The method of claim 15, wherein said SDG is (S,S)-SDG or (R,R)-SDG.

17. (canceled)

18. The method of claim 15, wherein said SDG is a synthetic SDG.

19. (canceled)

20. The method of claim 15, wherein said step of administering is performed orally or is performed intravenously.

21. (canceled)

22. (canceled)

23. The method of claim 15, wherein the subject is a human subject.

24. The method of claim 15, wherein said sepsis is associated with systemic inflammatory response syndrome (SIRS), infection, or multiple organ dysfunction syndrome.

25. The method of claim 15, wherein said sepsis is associated with a trauma or an injury or said sepsis is associated with a medical treatment.

26.-52. (canceled)

53. A method for reducing oxidative stress in cardiomyocytes of a subject having sepsis, comprising: administering to said subject an effective amount of secoisolariciresinol diglucoside (SDG), an analog thereof, a stereoisomer thereof, or a combination thereof, thereby reducing oxidative stress in said subject.

54. The method of claim 53, wherein said SDG is (S,S)-SDG or (R,R)-SDG.

55. (canceled)

56. The method of claim 53, wherein said SDG is a synthetic SDG.

57. (canceled)

58. The method of claim 53, wherein said step of administering is performed orally or is performed intravenously.

59. (canceled)

60. (canceled)

61. (canceled)

62. The method of claim 53, wherein said sepsis is associated with systemic inflammatory response syndrome (SIRS), infection, or multiple organ dysfunction syndrome.

63. The method of claim 53, wherein said sepsis is associated with a trauma or an injury or said sepsis is associated with a medical treatment.

64.-69. (canceled)
